SEQUENCE LISTING (88 sequences)

SEQ ID NO: 1 - protein, 16 aa, Artificial Sequence
  Synthetic Construct
LREVYGASLASYMERW

SEQ ID NO: 2 - protein, 18 aa, Artificial Sequence
  Synthetic Construct
AGLREVYGASLASYMERW

SEQ ID NO: 3 - protein, 18 aa, Artificial Sequence
  Synthetic Construct
AGLREVYGASLASYMERW

SEQ ID NO: 4 - protein, 19 aa, Artificial Sequence
  Synthetic Construct
CAGLREVYGASLASYMERW

SEQ ID NO: 5 - protein, 18 aa, Artificial Sequence
  Synthetic Construct
AGLREVYGASLASYMERW

SEQ ID NO: 6 - protein, 18 aa, Artificial Sequence
  Synthetic Construct
AGWREMYSALSASYVERL

SEQ ID NO: 7 - protein, 18 aa, Artificial Sequence
  Synthetic Construct
AGLREVYGASLASYMERW

SEQ ID NO: 8 - protein, 5 aa, Artificial Sequence
  Synthetic Construct
YMERW

SEQ ID NO: 9 - protein, 7 aa, Artificial Sequence
  Synthetic Construct
ASYMERW

SEQ ID NO: 10 - protein, 5 aa, Artificial Sequence
  Synthetic Construct
LREVY

SEQ ID NO: 11 - protein, 7 aa, Artificial Sequence
  Synthetic Construct
AGLREVY

SEQ ID NO: 12 - protein, 101 aa, Homo sapiens
MACPLEKALDVMVSTFHKYSGKEGDKFKLNKSELKELLTRELPSFLGKRTDEAAFQKLMSNLDSNRDNEVDFQEYCVFLSCIAMMCNEFFEGFPDKQPRKK

SEQ ID NO: 13 - protein, 338 aa, Homo sapiens
STVHEILCKLSLEGDHSTPPSAYGSVKAYTNFDAERDALNIETAIKTKGVDEVTIVNILTNRSNAQRQDIAFAYQRRTKKELASALKSALSGHLETLILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 14 - protein, 323 aa, Homo sapiens
HSTPPSAYGSVKAYTNFDAERDALNIETAIKTKGVDEVTIVNILTNRSNAQRQDIAFAYQRRTKKELASALKSALSGHLETLILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 15 - protein, 313 aa, Homo sapiens
VKAYTNFDAERDALNIETAIKTKGVDEVTIVNILTNRSNAQRQDIAFAYQRRTKKELASALKSALSGHLETLILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 16 - protein, 97 aa, Homo sapiens
MPSQMEHAMETMMFTFHKFAGDKGYLTKEDLRVLMEKEFPGFLENQKDPLAVDKIMKDLDQCRDGKVGFQSFFSLIAGLTIACNDYFVVHMKQKGKK

SEQ ID NO: 17 - protein, 5 aa, Artificial Sequence
  Synthetic Construct
YMERW

SEQ ID NO: 18 - protein, 5 aa, Artificial Sequence
  Synthetic Construct
LREVY

SEQ ID NO: 19 - protein, 5 aa, Artificial Sequence
  Synthetic Construct
WREMY

SEQ ID NO: 20 - protein, 5 aa, Artificial Sequence
  Synthetic Construct
YWERL

SEQ ID NO: 21 - protein, 16 aa, Artificial
  Synthetic Construct
WREMYSALSASYVERL

SEQ ID NO: 22 - protein, 18 aa, Artificial Sequence
  Synthetic Construct
AGWREMYSALSASYVERL

SEQ ID NO: 23 - protein, 5 aa, Artificial
  Synthetic Construct
YMERV

SEQ ID NO: 24 - protein, 5 aa, Artificial Sequence
  Synthetic Construct
YMERL

SEQ ID NO: 25 - protein, 5 aa, Artificial Sequence
  Synthetic Construct
YMERI

SEQ ID NO: 26 - protein, 5 aa, Artificial Sequence
  Synthetic Construct
YMERF

SEQ ID NO: 27 - protein, 5 aa, Artificial Sequence
  Synthetic Construct
YMERH

SEQ ID NO: 28 - protein, 5 aa, Artificial Sequence
  Synthetic Construct
YVERW

SEQ ID NO: 29 - protein, 5 aa, Artificial Sequence
  Synthetic Construct
YVERV

SEQ ID NO: 30 - protein, 5 aa, Artificial Sequence
  Synthetic Construct
YVERL

SEQ ID NO: 31 - protein, 5 aa, Artificial Sequence
  Synthetic Construct
YVERI

SEQ ID NO: 32 - protein, 5 aa, Artificial Sequence
  Synthetic Construct
YVERF

SEQ ID NO: 33 - protein, 5 aa, Artificial Sequence
  Synthetic Construct
YVERH

SEQ ID NO: 34 - protein, 5 aa, Artificial Sequence
  Synthetic Construct
YLERW

SEQ ID NO: 35 - protein, 5 aa, Artificial
  Synthetic Construct
YLERV

SEQ ID NO: 36 - protein, 5 aa, Artificial Sequence
  Synthetic Construct
YLERL

SEQ ID NO: 37 - protein, 5 aa, Artificial Sequence
  Synthetic Construct
YLERI

SEQ ID NO: 38 - protein, 5 aa, Artificial Sequence
  Synthetic Construct
YLERF

SEQ ID NO: 39 - protein, 5 aa, Artificial Sequence
  Synthetic Construct
YLERH

SEQ ID NO: 40 - protein, 5 aa, Artificial Sequence
  Synthetic Construct
YIERW

SEQ ID NO: 41 - protein, 5 aa, Artificial Sequence
  Synthetic Construct
YIERV

SEQ ID NO: 42 - protein, 5 aa, Artificial Sequence
  Synthetic Construct
YIERL

SEQ ID NO: 43 - protein, 5 aa, Artificial Sequence
  Synthetic Construct
YIERI

SEQ ID NO: 44 - protein, 5 aa, Artificial Sequence
  Synthetic Construct
YIERF

SEQ ID NO: 45 - protein, 5 aa, Artificial Sequence
  Synthetic Construct
YIERH

SEQ ID NO: 46 - protein, 5 aa, Artificial Sequence
  Synthetic Construct
FMERW

SEQ ID NO: 47 - protein, 5 aa, Artificial Sequence
  Synthetic Construct
FMERV

SEQ ID NO: 48 - protein, 5 aa, Artificial Sequence
  Synthetic Construct
FMERL

SEQ ID NO: 49 - protein, 5 aa, Artificial Sequence
  Synthetic Construct
FMERI

SEQ ID NO: 50 - protein, 5 aa, Artificial Sequence
  Synthetic Construct
FMERF

SEQ ID NO: 51 - protein, 5 aa, Artificial Sequence
  Synthetic Construct
FMERH

SEQ ID NO: 52 - protein, 5 aa, Artificial Sequence
  Synthetic Construct
FVERW

SEQ ID NO: 53 - protein, 5 aa, Artificial Sequence
  Synthetic Construct
FVERV

SEQ ID NO: 54 - protein, 5 aa, Artificial Sequence
  Synthetic Construct
FVERL

SEQ ID NO: 55 - protein, 5 aa, Artificial Sequence
  Synthetic Construct
FVERI

SEQ ID NO: 56 - protein, 5 aa, Artificial Sequence
  Synthetic Construct
FVERF

SEQ ID NO: 57 - protein, 5 aa, Artificial Sequence
  Synthetic Construct
FVERH

SEQ ID NO: 58 - protein, 5 aa, Artificial Sequence
  Synthetic Construct
FLERW

SEQ ID NO: 59 - protein, 5 aa, Artificial Sequence
  Synthetic Construct
FLERV

SEQ ID NO: 60 - protein, 5 aa, Artificial Sequence
  Synthetic Construct
FLERL

SEQ ID NO: 61 - protein, 5 aa, Artificial Sequence
  Synthetic Construct
FLERI

SEQ ID NO: 62 - protein, 5 aa, Artificial Sequence
  Synthetic Construct
FLERF

SEQ ID NO: 63 - protein, 5 aa, Artificial Sequence
  Synthetic Construct
FLERH

SEQ ID NO: 64 - protein, 5 aa, Artificial Sequence
  Synthetic Construct
FIERW

SEQ ID NO: 65 - protein, 5 aa, Artificial Sequence
  Synthetic Construct
FIERV

SEQ ID NO: 66 - protein, 5 aa, Artificial Sequence
  Synthetic Construct
FIERL

SEQ ID NO: 67 - protein, 5 aa, Artificial Sequence
  Synthetic Construct
FIERI

SEQ ID NO: 68 - protein, 5 aa, Artificial Sequence
  Synthetic Construct
FIERF

SEQ ID NO: 69 - protein, 5 aa, Artificial Sequence
  Synthetic Construct
FIERH

SEQ ID NO: 70 - DNA, 912 bp, Homo sapiens
  CDS (1)..(912)
atgccgctgctgctactgctgcccctgctgtgggcaggggccctggctatggataagcttgctagcggtaccctcgaggatatctctagaccagtgtgctgggcggccgcggatccaggactgcgcgaggtgtacggagcgggccttgccgggtacatggagcgctgggctagcggaggggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 71 - protein, 303 aa, Homo sapiens
MPLLLLLPLLWAGALAMDKLASGTLEDISRPVCWAAADPGLREVYGAGLAGYMERWASGGDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 72 - DNA, 879 bp, Homo sapiens
  CDS (1)..(879)
atgccgctgctgctactgctgcccctgctgtgggcaggggccctggctatggataagcttgctagcggtaccctcgaggatatctctagaccagtgtgctgggcggccgcggatccaggactgcgcgaggtgtacgctagcggaggggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 73 - protein, 292 aa, Homo sapiens
MPLLLLLPLLWAGALAMDKLASGTLEDISRPVCWAAADPGLREVYASGGDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 74 - DNA, 879 bp, Homo sapiens
  CDS (1)..(879)
atgccgctgctgctactgctgcccctgctgtgggcaggggccctggctatggataagcttgctagcggtaccctcgaggatatctctagaccagtgtgctgggcggccgcggatccaggatacatggagcgctgggctagcggaggggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 75 - protein, 292 aa, Homo sapiens
MPLLLLLPLLWAGALAMDKLASGTLEDISRPVCWAAADPGYMERWASGGDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 76 - DNA, 26 bp, Artificial Sequence
  Primer
ctggatcctgtcatggcgtgccctct

SEQ ID NO: 77 - DNA, 23 bp, Artificial Sequence
  Primer
agggatccaaccacatcagagga

SEQ ID NO: 78 - DNA, 34 bp, Artificial Sequence
  Primer
gcggatccccgtctactgttcacgaaatcctgtg

SEQ ID NO: 79 - DNA, 33 bp, Artificial Sequence
  Primer
tgcggtcgacgtcatctccaccacacaggtaca

SEQ ID NO: 80 - DNA, 31 bp, Artificial Sequence
  Primer
gcggatcccctcactctacacccccaagtgc

SEQ ID NO: 81 - DNA, 36 bp, Artificial Sequence
  Primer
gcggatccccggtcaaagcctatactaactttgatg

SEQ ID NO: 82 - DNA, 29 bp, Artificial Sequence
  Primer
gcggatccatgccatctcaaatggaacac

SEQ ID NO: 83 - DNA, 31 bp, Artificial Sequence
  Primer
ccgctcgagttctgcctacttctttcccttc

SEQ ID NO: 84 - DNA, 69 bp, Artificial Sequence
  Primer
gatccaggactgcgcgaggtgtacggagcgggccttgccgggtacatggagcgctgggctagcggaggg

SEQ ID NO: 85 - DNA, 21 bp, Artificial Sequence
  Primer
tgctgggcggccgcggatcca

SEQ ID NO: 86 - DNA, 26 bp, Artificial Sequence
  Primer
ttatgcacctccacgccgtccacgta

SEQ ID NO: 87 - DNA, 30 bp, Artificial Sequence
  Primer
caccatgccgctgctgctactgctgcccct

SEQ ID NO: 88 - DNA, 35 bp, Artificial Sequence
  Primer
agctcatttacccggagacagggagaggctcttct

(19) United States
(12) Patent Application Publication
(10) Pub. No.: US 2005/0277575 A1
(43) Pub. Date: Dec. 15, 2005

(54) Therapeutic compositions and methods for treating diseases that involve angiogenesis

(76) Inventors: Alexandre Semov, Dorval (CA); Anatoli Onichtchenko, Pointe Claire (CA); Ludmila Iourtchenko, Pointe Claire (CA); Benoit Ochietti, Pierre Fonds (CA); Grzegorz Pietrzynski, Montreal (CA); Valery Alakhov, Baie d'Urfe (CA)

Correspondence Address: STITES & HARBISON PLLC, 1199 NORTH FAIRFAX STREET, SUITE 900, ALEXANDRIA, VA, 22314, US

(21) Appl. No.: 11/087,719

(22) Filed: Mar. 24, 2005

Related U.S. Application Data
(60) Provisional application No. 60/555,668, filed on Mar. 24, 2004.

(51) Int. Cl. A61K 38/17; A61K 48/00

(52) U.S. Cl. 514/2; 514/44; 536/23.1; 435/320.1; 435/325

(57) ABSTRACT

According to the present invention, S100A4 protein also known as Mts-1 interferes with the function of Annexin 2 and Annexin2/P11 tetramer by binding to Annexin 2. The present inventors have demonstrated that binding of S100A4 with Annaxin 2 modulates angiogenesis by interfering with Annexin 2 mediated tissue plasminogen activator (tPA) dependant conversion of plaminogen into plasmin and further conversion of plasmin into angiostatins. The present invention has further identified that the S100A4 protein binds to the N-terminal region of Annexin 2. Accordingly, the present invention discloses peptides and pharmaceutical compositions thereof and methods of treating cancers and other diseases that involve angiogenesis, by interfering with the interaction between S100A4 and Annexin

### FIELD OF THE INVENTION

This invention relates to therapeutic agents to treat cancer and other diseases that involve angiogenesis. More particularly, this invention relates to the interference with the S100A4 protein binding to Annexin 2 and its complexes with other proteins that are involved in Annexin-2 angiogenesis control, which interference modulates angiogenesis.

### BACKGROUND OF THE INVENTION

1. Mts-1/S100A4 Protein

Mts-1/S100A4 protein belongs to the S100 family of Ca-binding proteins, these proteins contain two EF-hands, Ca-binding motifs, consisting of 12 and 14 amino acids. S100 proteins were initially characterized as low-weight (10 to 12 kDa) acidic proteins and named by their solubility in 100% ammonium sulfate. Members of the S100 family have been implicated in a variety of cellular events, including growth, signaling, differentiation, and motility, however, much interest has concentrated on S100A4 and several other S100 protein family members, such as S100A2, S100A6, and S100B for their potential relevance in neoplastic diseases. Human S100A4 is a protein of 101 amino acids with a molecular weight of 11.5 kDa. It has been described under a variety of names including p9Ka, calvasculin, or CAPL. The corresponding gene is known as mts1 (metastasin), pEL98, 18A2, 42A, and fsp1 (fibroblast-specific protein 1).

In humans, S100A4 protein expression has been demonstrated in activated monocytes, macrophages, and polymorphonuclear granulocytes, as well as in bone marrow and spleen (Takenaga et al., Biochem Biophys Res Commun 1994, 202:94-101). Low levels of S100A4 protein have been detected only in subsets of cells of normal ovary and prostate tissues, and it has not been detected in other normal tissues including those obtained from the breast, colon, thyroid, lung, kidney, and pancreas (Ilg et al., Int J Cancer 1996, 68:325-332). In rats, relatively high expression of S100A4 was registered in smooth muscles and endothelial cells (Gibbs et al., J Histochem Cytochem 1995, 43:169-180). An increasing body of evidence clearly indicates that, in addition to its intracellular location, protein S100A4 may be secreted into the extracellular space. For instance, studies on the rat mammary gland suggested an extracellular location of S100A4 around the ductuli (Gibbs et al., J Histochem Cytochem 1995, 43:169-180). Further, release of protein S100A4 has been reported to occur in intact periodontal cultured cells (Duarte et al., Biochem Biophys Res Commun 1999, 255:416-420) and mammary carcinoma cells (Ambartsumian et al., Invasion Metastasis 1998, 18:96-104). These findings are in agreement with the notion that many other S100 proteins can be secreted.

The data collected so far indicate that most of S100 proteins form noncovalent dimer inside the cell and covalently linked dimers in the extracellular space (Zimmer et al., Brain Res Bull 1995, 37:417-429). Presumably, calcium binding to these proteins induces conformational changes resulting in exposure of new binding sites at their surface, and, consequently, allows for the interaction with novel target proteins. Recent studies also demonstrated that, in solution, S100A4 exists in a monomer-dimer equilibrium influenced by the binding of calcium (Tarabykina et al., J Biol Chem 2001, 276:24212-24222) and that S100A4 homo- and heterodimerization may occur in vivo (Tarabykina et al., FEBS Lett 2000, 475:187-191).

Multiple observations support a role of protein S100A4 in invasive growth and in metastasis of cancers. Transfection experiments showed that rodent or human S100A4 can induce a metastatic phenotype in previously nonmetastatic rat mammary cells (Davies et al., Oncogene 1993, 8:999-1008; Lloyd et al., Oncogene 1998, 17:465-473). Similarly, transfection of the rodent S100A4 gene into the B 16 murine melanoma (Parker et al., DNA Cell Biol 1994, 13:1021-1028) and into human breast cancer MCF-7 cells (Grigorian et al., Int J Cancer 1996, 67:831-841) increased the capability to metastasize to the lungs. Conversely, antisense S100A4 RNA or anti-S100A4 ribozyme suppressed the metastatic potential of highly metastatic cell lines (Maelandsmo et al., Cancer Res 1996, 56:5490-5498; Takenaga et al., Oncogene 1997, 14:331-337) Transgenic mouse studies demonstrated that protein S100A4 by itself was not able to initiate tumors. However, it induced metastatic disease of cells that had been initiated by other oncogenes (Ambartsumian et al., Oncogene 1996, 13:1621-1630; Davies et al., Oncogene 1996, 13:1631-1637) Gene expression in cultivated tumor cells and in xenograft tumors in mice could be different. Genes not expressed in cultivated cells can be induced in the tumor environment in vivo if the growing tumor needs them for its progression. Keeping this in mind, it is important to mention that indeed overexpression of S100A4 was detected in tumors induced in mice by subcutaneous transplantation of human cancer cell lines in comparison with original cell lines cultivated in vitro (Creighton et al., Genome Biol. 2003; 4(7):R46).

Mts-1 is strongly overexpressed in many human cancers of various origins. It is a well-established marker of tumor progression, invasion, metastasis formation, as well as poor survival prognosis. Two retrospective studies, based on the same well-characterized group of 349 patients with a follow-up period of 19 years (Platt-Higgins et al., Int J Cancer 2000, 89:198-208; Rudland et al., Cancer Res 2000, 60:1595-1603), analyzed the prognostic significance of protein S100A4 in breast cancer and evaluated the association between protein expression, as detected by immunohistochemical staining, and variables with potential prognostic value for patient outcome. The antiserum stained 56% of the carcinomas either strongly or at a borderline level, whereas 44% of the carcinomas remained unstained. The overall survival for patients with carcinomas expressing S100A4 was significantly worse than for those patients considered negative for S100A4. In analogous studies the prognostic significance of protein S100A4 expression has recently been evaluated in a series of esophageal-squamous carcinomas, non-small lung cancers, and primary gastric cancers. (Ninomiya et al., Int J Oncol 2001, 18:715-720; Kimura et al., Int J Oncol 2000, 16:1125-1131; Yonemura et al., Clin Cancer Res 2000, 6:4234-4242) Patients with S100A4-positive esophageal carcinomas [13 of 52 (25%)] had a significantly poorer prognosis than patients with S100A4-negative carcinomas; the protein S100A4 status in cancer specimens remained the only independent prognostic parameter in a multivariate analysis. Immunohistochemically S100A4 was detectable in 81 of 135 (60%) lung cancers. S100A4 was found to be useful to identify patients with poor prognosis, as its tissue expression was correlated with progression of the tumor size as well as nodal status (Kimura et al., Int J Oncol 2000, 16:1125-1131). Finally, protein S100A4 was found to be significantly more expressed in poorly than in well-differentiated gastric adenocarcinomas [51 of 92 (55%)], and was correlated with nodal metastatic disease and peritoneal dissemination (Yonemura et al., Clin Cancer Res 2000, 6:4234-4242). Immunohistochemical studies revealed no staining for protein S100A4 in the epithelial cells of normal colonic mucosa and in colonic adenomas [0 of 12], whereas carcinomas arising in adenomas and invasive carcinomas showed S100A4 expressing cells in 44% [8 of 18] and 94% [50 of 53] of cases, respectively (Takenaga et al., Clin Cancer Res 1997, 3:2309-2316). In pancreatic cancer Rosty et al. (Am J Pathol 2002, 160:45-50) did not find the S100A4 expression in low-grade intraepithelial neoplasia lesions [0 of 69], low level of expression was detected in high-grade pancreatic neoplasia lesions [3 of 18 (17%)], but most of pancreatic invasive carcinomas expressed S100A4 [57 of 61 (93%)]. Expression of S100A4 was also associated with metastasis and poor survival in patients with bladder cancer (Davies, Histol Histopathol. 2003 July; 18(3):969-80).

Recently, up-regulation of S100A4 expression was reported for several pathologies associated with activated endothelium such as ocular neovascularization, artheriopathy, inflammation and fibrosis. S100A4 mRNA was rarely detected in keratocytes or epithelial cells of the normal rabbit cornea. Likewise, S100A4 antigen was not found in normal mouse corneas. However, after removal of the corneal epithelium, fibroblasts are activated and had readily detectable S100A4 expression. In fact, S100A4 mRNA was identified as the most abundant message present in regenerating cornea. Its expression and distinct subcellular redistribution suggest that S100A4 may be involved in the interconversions that occur between keratocytes, fibroblasts, and myofibroblasts during corneal wound healing (Ryan et al., Invest Ophthalmol Vis Sci. 2003 October; 44(10):4255-62). In another experiments approximately 5% of transgenic mice overexpressing S100A4/Mtsl develop pulmonary arterial changes resembling human plexogenic arteriopathy with intimal hyperplasia leading to occlusion of the arterial lumen. In surgical lung biopsies from children with pulmonary hypertension secondary to congenital heart disease, S100A4/Mtsl was not detected in arteries with low-grade hypertensive lesions but was expressed in smooth muscle cells of lesions showing neointimal formation with highest expression in vessels with an occlusive neointima and plexiform lesions (Greenway et al., Am J Pathol. 2004 January; 164(1):253-62). The role for S100A4 in inflammation follows from overexpression of the protein in synovial tissues from patients with rheumatoid arthritis, while it was not expressed in normal individuals (Masuda et al., Arthritis Res. 2002; 4(5):R8). Important role for S100A4 in tissue fibrosis follows from the experiments demonstrating that the induction of this protein is a crucial event in epithelial-mesenchymal transformation. Mesenchymal cells have the ability, which true epithelial cells do not, to invade and migrate through the extracellular matrix. This transformation can be induced by transforming growth factor beta 1 (TGF-bl) or epidermal growth factor (EGF) and antisense oligonucleotides to S100A4/FSP1 were shown to block this transformation (Okada et al., Am J. Physiol. 1997 October; 273(4 Pt 2):F563-74). Futhermore, FSP1-expressing fibroblasts produced by local epithelial-mesenchymal transformation constituted the main population of fibroblasts arising during experimental renal fibrosis in transgenic mice (Iwano et al., J Clin Invest. 2002 August; 110(3):341-50). The processes of metastasis and epithelial-mesenchymal transformation probably have some common molecular programs. It was proposed that transition of cancer cells to motile phenotype is a result of molecular exaptation (Xue et al., Cancer Res. 2003 Jun. 15; 63(12):3386-94). Molecular exaptation is a form of economy by which cells reuse known physiological processes to provide new functions. Epithelial cells that normally use FSP1-directed epithelial-mesenchymal transformation to become fibroblasts rely on the same molecular program to metastasize when they convert from in situ to invasive tumor cells.

Usually S100A4 is an intracellular protein, where it is thought to function in modulation of its target proteins properties, first of all their phosphorylation. S100A4 binds p53 tumor suppressor protein (Chen et al., Biochem Biophys Res Commun. 2001 Sep. 7; 286(5):1212-7) and affects the phosphorylation of p53 by PKC and modulates the expression of p53-regulated genes, such as p21/WAF1 and bax (Grigorian et al., J Biol. Chem. 2001 Jun. 22; 276(25):22699-708). It was concluded that S100A4 cooperates with wild-type p53 to stimulate apoptosis, and that this process, at an early stage of tumor development, may accelerate the loss of wild-type p53 functions, and consequently lead to the selection of more aggressive cell clones. Interaction of S100A4 with MetAP2 (Methionin aminopeptidase 2), a well known target for potent antiangiogenic inhibitors, fumagillin and ovalicin, was demonstrated in murine endothelial cells (Endo et al., J Biol Chem. 2002 Jul. 19; 277(29):26396-402). An analog of fumagillin, known as TNP-470 or AGM-1470, which has been undergoing clinical trials for treating a variety of cancers, induces the activation of the p53 pathway, causing an accumulation of the p21/WAF1 and blocking endothelial cell cycle progression (Zhang et al., Proc Natl Acad Sci USA. 2000 Jun. 6; 97(12):6427-32). Binding of S100A4 to both p53 and MetAP2 can link together apoptotic and angiogenic pathways in tumor development. Binding in vivo of S100A4 and Liprin beta 1 was detected in mouse mammary adenocarcinoma cell lines. Both proteins colocolized in perinuclear regions and at the protrusion sites of the plasma membrane. S100A4-liprin beta 1 interaction resulted in the inhibition of liprin phosphorylation by PKC and protein kinase CK II in vitro (Kriajevska et al., J Biol. Chem. 2002 Feb. 15; 277(7):5229-35). The role of S100A4 in motility has been shown through the characterization of its interactions with a critical cytosceletal component of motility, nonmuscle myosin. S100A4 binds specifically to non-muscle myosin heavy chain IIA, disrupts myosin self-assembly, and inhibits PKC or CK II-dependent phosphorylation of the heavy chain (Kriajevska et al., Biochim Biophys Acta. 2000 Dec. 20; 1498(2-3):252-63). Interaction with another component of the cytoskeleton, non-muscle tropomyosin, was also reported and might be responsible for the disorganization of actin filaments too (Takenaga et al., J. Cell Biol. 1994 March; 124(5):157-68). Another recently discovered binding partner for S100A4 is CCN3NOV/IGFBP9 (Nephroblastoma overexpressed gene) (Li et al., Mol Pathol. 2002 August; 55(4):250-61). This is a putative secreted protein with similarity to insulin-like growth factor-binding proteins. Aberrant expression of NOV is associated with the development of several tumors of different origin but exact mechanisms of its function remain obscure (Manara et al., Am J Pathol. 2002 March; 160(3):849-59).

Most of the S100 proteins have been shown to share the ability to homodimerize and/or heterodimerize with other S100 family members. Heterodimerization of S1004 and S100A1 was shown in vitro and in vivo in the yeast two-hybrid system (Wang et al., J Biol. Chem. 2000 Apr. 14; 275(15):11141-6; Tarabykina et al., FEBS Lett. 2000 Jun. 23; 475(3):187-91). Partial colocalization of S100A4 and S100A1 was detected on stress fibers and in the perinuclear region (Wang et al., J Biol. Chem. 2000 Apr. 14; 275(15):11141-6). An S100A4 mutant that is deficient for calcium binding retains the ability to form homodimers, suggesting that S100A4 can exist as calcium-free or calcium-bound dimers in vivo (Kim and Helfinan, J Biol Chem. 2003 Aug. 8; 278(32):30063-73). However, a calcium-bound S100A4 monomer only interacts with another calcium-bound monomer and not with an S100A4 mutant that does not bind calcium. Interestingly, despite the calcium dependence for interaction with known protein partners, calcium binding is not necessary for localization to lamellipodia. Both wild type and a mutant that is deficient for calcium binding colocalize with known markers of actively forming leading edges of lamellipodia, Arp3 and neuronal Wiskott-Aldrich syndrome protein.

As it was mentioned earlier, S100A4 is detected mainly in cytoplasm, in perinuclear regions, on stress fibers and in leading edges. However, in some conditions S100M can be secreted into the extracellular space. Up to 20% of S100A4 can be released by S100A4-overexpressing tumor cells and detected in conditioned medium (Ambartsumian et al., Oncogene. 2001 Aug. 2; 20(34):4685-95). S100A4 was also secreted by periodontal ligament cells both in vivo and in vitro (Duarte et al., Biochem Biophys Res Commun. 1999 Feb. 16; 255(2):416-20). Furthermore, this protein was found in the serum of normal mice and in plasma of cancer patients with high expression of S100A4 in tumor (Ambartsumian et al., Oncogene. 2001 Aug. 2; 20(34):4685-95).

Effects of extracellular S100A4 were also demonstrated. Oligomeric but not dimeric forms of S100A4 strongly induced differentiation of cultured hippocampal neurons. MtsI-stimulated neurite outgrowth involves activation of phospholipase C and protein kinase C, depends on the intracellular level of Ca(2+) and requires activation of the extracellular signal-regulated kinases (ERKs) 1 and 2 (Novitskaya et al., J Biol Chem. 2000 Dec. 29; 275(52):41278-86). Extracellular S100A4 stimulated the migration of astrocytic tumor cells. S100A4 treatment decreased the amount of polymerized F-actin and down regulated expression of RhoA, Cdc42, and N-WASP (Belot et al., Biochim Biophys Acta. 2002 Nov. 4; 1600(1-2):74-83).

It was demonstrated in in vitro and in vivo studies that S100A4 protein interacts with endothelial tissues and induces angiogenesis. Tumors developing in S100A4 transgenic mice revealed an increased vascular density. S100A4 oligomers were capable of stimulating motility of endothelial cells in vitro and inducing corneal neovascularization in vivo (Ambartsumian et al., Oncogene. 2001 Aug. 2; 20(34):4685-95). Experiments with S100A4-inducible cell lines suggest that S100A4 strongly down-regulates the thrombospondin 1 (THBS1) gene (Roberts, FASEB J 1996, 10:1183-1191), which is known to repress tumor progression by inhibition of angiogenesis. Thus, it is conceivable that, at least partially, S100A4 promotes angiogenesis in vivo by preventing the anti-angiogenic effect of THBS1. Although effects of S100A4 on endothelial cells have been reported and its pro-angiogenic effect has been demonstrated, direct binding of S100A4 to endothelial cells was not shown and putative cell membrane receptors for extracellular forms of the S100A4 protein are currently unknown.

II. Annexin II.

Annexins are a family of structurally related proteins whose common property is calcium-dependent binding to phospholipids located on the cytosolic face of the plasma membrane. Binding to calcium and phospholipids is mediated by highly conserved annexin repeats, each annexin contains 4 or 8 such domains. Specificity and diversity of each of annexins is carried by the N-terminal domain, different for all annexins, but may also be the consequence of interactions of annexins either with other members of this protein family or other cellular partners. Annexins participate in membrane trafficking events such as exocytosis, endocytosis and cell-to-cell adhesion. They may provide a major pathway for communication between plasma membrane phospholipids and the cytoskeleton.

Annexin II has been shown to exist as a monomer, homodimer or heterotetramer with p11 protein that belongs to the S100 protein family and is also known as S100A10. Annexin is a major substrate for protein kinases including the PDGF-receptor, src protein tyrosine kinase, and protein kinase C but functional role of Annexin II phosphorylation is not clear yet (Bellagamba et al., J Biol. Chem. 1997 Feb. 7; 272(6):3195-9). It was shown that Annexin II binds to F-actin and the binding site was mapped in its C-terminal end (Filipenko and Waisman, J Biol. Chem. 2001 Feb. 16; 276(7):5310-5).

Overexpression of annexin II has been reported in various carcinomas. Annexin II is overexpressed in primary colorectal carcinomas (31 cases of 105, 29.5%) and its overexpression correlated with histologic type, tumor size, depth of invasion and poor prognosis. Expression of annexin II correlated significantly with that of tenascin-C. Tenascin-C is a ligand for extracellular Annexin II (see below) that also has been reported to be a prognostic marker for several carcinomas (Emoto et al., Cancer. 2001 Sep. 15; 92(6): 1419-26). Autoantibodies against Annexin II were detected in 60% of patients with lung adenocarcinoma and 33% of patients with squamous cell lung carcinoma but not in the noncancer controls (Brichory et al., Proc Natl Acad Sci USA. 2001 Aug. 14; 98(17):9824-9). In case of gastric carcinoma, thirty-three percent of 153 cases were immunopositive for Annexin II, overexpression of which was more frequent in lymph node, metastasis and venous invasion. Annexin II and c-erbB-2 overexpression were significantly correlated and patients with Annexin II had poorer prognoses (Emoto et al., Anticancer Res. 2001 March-April; 21(2B): 1339-45). The direct role for Annexin II in metastasis was demonstrated in the following experiments. Over-expression of Annexin II and activated leukocyte cell adhesion molecule (ALCAM) was detected during the metastasis progression after chemotherapy with Adriamycin. Metastasis to the lung was observed in the mice when cells over-expressing Annexin II and ALCAM had been inoculated via tail vein (Choi et al., Cli E Metastasis. 2000; 18(1):45-50). Using a proteomics approach, overexpression of 3 proteins, Annexin II, Annexin I and enolase-alpha, was identified in head and neck squamous cell carcinoma cell lines established from metastatic lymph nodes in comparison with cell lines established from the primary tumor from the same patient (Wu et al., Clin Exp Metastasis. 2002; 19(4):319-26). Overexpression of Annexin II was detected in endothelial cells forming tubes in 3D collagen gels, compared to migrating and proliferating cells in 2D cultures implicating its role in vascular remodeling and angiogenesis (Aitkenhead et al., Microvasc Res. 2002 March; 63(2):159-71).

Although Annexin II lacks a signal peptide and its mechanism of secretion is unknown, extracellular annexin II has been found in several tissues as both soluble and membrane-bound protein. Besides cancer, extracellular annexin II may be important in several biological processes, such as fibrinolysis, cell adhesion, ligand-mediated cell signaling, and virus infection (Siever and Erickson. Int J Biochem Cell Biol. 1997 November; 29(11):1219-23). The Annexin II/S100A10 tetramer has been located on the extracellular surface of endothelial and metastatic cancer cells and was shown to mediate binding of metastatic cells to the endothelium (Waisman, Mol Cell Biochem. 1995 August-September; 149-150:301-22). Cell-surface annexin II has been identified as a receptor for a number of ligands. Annexin II may serve as a membrane receptor for rapid actions of 1 alpha, 25-dihydroxyvitarnin D(3) and binding to vitamin D(3) was inhibited by calcium (Baran et al., J Cell Biochem. 2000 Oct. 20; 80(2):259-65). High affinity binding of beta 2-glycoprotein I (beta(2)GPI) to human endothelial cells was shown to be mediated by annexin II (Ma et al., J Biol. Chem. 2000 May 19; 275(20):15541-8). Beta(2)GPI) is an abundant plasma phospholipid-binding protein and an autoantigen in the antiphospholipid antibody syndrome, it is also a component of atherosclerotic plaques. Binding of beta(2)GPI to endothelial cells targets them for activation by anti-beta(2)GPI autoantibodies, which circulate and are associated with thrombosis in patients with the antiphospholipid antibody syndrome. Another important ligand for Annexin II is cathepsin B. Cathepsin B is a lysosomal cysteine protease in normal cells and tissues. Experimental and clinical evidence has linked cathepsin B with tumor invasion and metastasis. In malignant tumors and premalignant lesions, the expression of cathepsin B is highly unregulated and the enzyme is secreted and becomes associated with the surface of tumor cells via Annexin II/p11 tetramer. This binding seems to facilitate conversion of procathepsin B to its active forms. Cathepsin B and the annexin II heterotetramer colocalize in caveolae (lipid raft) fractions isolated from tumor cells where it can degrade extracellular matrix proteins, such as tenascin-C, collagen IV and laminin, and can activate the precursor form of urokinase plasminogen activator (uPA), perhaps thereby initiating an extracellular proteolytic cascade (Mai et al., J Biol Chem. 2000 Apr. 28; 275(17):12806-12; Roshy, Sloane, and Moin. Cancer Metastasis Rev. 2003 June-September; 22(2-3):271-86). Tenascin-C is an extracellular matrix glycoprotein with predominantly antiadhesive properties that also has been reported to be a prognostic marker for several carcinomas. Selective expression of tenascin-C in tumors has led to the development of radio-labelled monoclonal anti-tenascin-C antibodies for targeting tumor therapy (Mackie, Int I Biochem Cell Biol. 1997 October; 29(10):1133-7). Tenascin-C induced loss of focal adhesion and produced mitogenic response in confluent endothelial cells, as well as enhanced cell migration in a cell culture wound assay. Antibodies to Annexin II blocked all three cellular responses to Tenascin-C (Chung, Murphy-Ullrich, and Erickson HP. Mol Biol Cell. 1996 June; 7(6):883-92).

Annexin II/S100A10 complex was recently identified as a co-receptor for plasminogen and tissue plasminogen activator (t-PA) on the surface of endothelial cells (Hajjar, Jacovina, and Chacko. J Biol Chem. 1994 Aug. 19; 269(33):21191-7) and was found to facilitate generation of plasmin (Kwon et al.,. J Biol Chem. 2002 Mar. 29; 277(13):10903-11) and its further conversion into its anti-angiogenic fragments also known as angiostatins (Tuszynski et al., Microvasc Res. 2002 November; 64(3):448-62). Annexin II, when expressed on the surface of cultured macrophages, promotes their ability to remodel extracellular matrix through tPA-dependent generation of cell surface plasmin (Brownstein et al., Blood. 2004 Jan. 1; 103(1):317-24). The abundant presence of annexin-2 on the surface of cancer cells may contribute to their invasive potential through extracellular matrix either by generating plasmin or, by plasmin-mediated proteolytic activation of other metalloproteinases. On the other hand, by increasing the pool of plasmin, a precursor to an important anti-angiogenic factor angiostatin, Annexin II could play a pivotal physiological role in the pro- and anti-angiogenic switch mechanism.

III. S100—Annexin Protein Interactions.

Multiple interactions between various Annexins and S100 proteins in vivo and in vitro have been previously reported. Binding and heterotetramer formation was shown in Annexin I—S100A11, Annexin 11—S100A6, Annexin 6—S100A1, Annexin 6—S100A6, Annexin 6—S100B, Annexin V—S100A1, Annexin V—S100B, Annexin II—S100A6, and in Annexin II-S100A10 pairs. Moreover, heterodimer formation was shown between different annexins, such as Annexin I and Annexin II, and between different representatives of S100 family, such as S100A4 and S100A1, S100A1 and S100B, S100A6 and S100B, S100A8 and S100A9, S100A11 and S100B.

No interaction between S1004A and Annexin II described herein has been previously reported and is entirely unexpected.

### BRIEF DESCRIPTION OF THE INVENTION

One aspect of the invention is directed to methods of identifying a compound which interferes with the interaction between S100A4 protein and Annexin 2 by binding to S100A4 or Annexin 2.

Compounds so identified by the screening methods of the present invention form another aspect of the present invention.

An aspect of the invention includes an isolated polypeptide selected from the group consisting of:
(a) an amino acid sequence X¹X²ERX³, wherein X¹ is selected from the group consisting of Y and F; X² is selected from the group consisting of M, V, L, I; and X³ is selected from the group consisting of W, V, L, I, F, H; or a derivative thereof; and
(b) an amino acid sequence X RE X²X³, wherein X¹ is selected from the group consisting of W, V, L, I, F, H; X² is selected from the group consisting of M, V, L, I; and X³ is selected from the group consisting of Y and F; or a derivative thereof.

Another aspect of the invention are compounds comprising at least two polypeptides as noted above conjoined with a chemical linker, the chemical linker being between approximately 5 Angstroms and approximately 100 Angstroms.

Yet another aspect of the invention are compositions comprising a polypeptide noted above or a compound noted above and a pharmaceutically or physiologically acceptable carrier.

Still yet another aspect of the present invention are methods of treating a disease associated with activated endothelium in a patient in need of such therapy comprising administering to said patient a therapeutically effective amount of the compositions noted above.

Other aspects of the invention include nucleic acid sequences encoding the polypeptides and/or compounds of the present invention as well as expression vectors containing the nucleic acid sequences and host cells transfected with the expression vectors.

Another embodiment of the present invention provides a method for interfering with the binding between S100A4 and Annexin 2 in a subject by administering to the subject, an effective amount of a compound that modulates the interaction between S100A4 and Annexin 2 by binding to S100A4 or Annexin 2. For example, one such compound comprises one or more of the peptides that modulate interaction between S100A4 and Annexin 2 such as those listed in Table 1; their fragments or polypeptide and non-polypeptide derivatives of such peptides are also within the scope of the present invention.

The invention further provides a pharmaceutical composition comprising at least one peptide or derivative thereof, wherein said polypeptide or derivative thereof is capable of specific binding with S100A4 or Annexin 2. Such a polypeptide is herein referred to as a Ligand.

The pharmaceutical composition of the present invention comprising at least one peptide or derivative thereof, wherein said polypeptide or derivative thereof that is capable of specific binding with the high affinity S100A4 or Annexin 2 further comprises the ability to modulate the interaction of S100A4 with Annexin 2 and modulates biological effects mediated by the above interaction.

A preferred peptide of the present invention provides the amino acid sequence of SEQ ID NOs 5, 12, 14, 33, 34, 35, 36, or 37 wherein the peptide is a ligand of S100A4 or Annexin 2 and modulates the interaction between S100A4 and Annexin 2.

The present invention also provides analogs of the pharmaceutical composition which can comprise in its molecular structure residues being derivatives of compounds other than amino acids, referenced herein as “peptide mimetics” or “peptidomimetics”. Other analogs of the peptide Ligand are compounds having changed topology of its chain, in particular nonlinear compounds, which have chemical bonds that close cycle or cycles in the molecule and constrain its structure.

The peptide Ligand of the present invention can be made by using well-known methods including recombinant methods and chemical synthesis. Recombinant methods of producing a peptide through the introduction of a vector including nucleic acid encoding the peptide into a suitable host cell is well known in the art, such as is described in Sambrook et al., Molecular Cloning: A Laboratory Manual, 2d Ed, Vols. 1 to 8, Cold Spring Harbor, N.Y. (1989), which is herein incorporated by reference. A linear sequence is synthesized, for example, by the solid phase peptide synthesis of Merrifield et al., J. Am. Chem. Soc., 85:2149 (1964), which is incorporated herein by reference). Alternatively, a peptide ligand of the present invention can be synthesized using standard solution methods well known in the art (see, for example, Bodanszky, M., Principles of Peptide Synthesis (Springer-Verlag, 1984)), which is herein incorporated by reference). Newly synthesized peptide ligands can be purified, for example, by high performance liquid chromatography (HPLC), and can be characterized using, for example, mass spectrometry or amino acid sequence analysis. Although a purity of greater than 95 percent for the synthesized peptide is preferred, lower purity may be acceptable. The analogs of the peptide Ligand can be peptides with altered sequences comprising another selection of L-α-amino acid residues, D-α-amino acid residues and non-α-amino acid residues.

Therefore, in another embodiment, the pharmaceutical composition of the present invention provides derivatives of peptides SEQ IDs. NO.: 5-15, 33-38 that are comprised of oligopeptides, chemical derivatives or peptidomimetics that are capable of specific binding with S100A4 or Annexin 2.

In another embodiment of the invention, the Ligand is associated with a biological agent. Such an association can be achieved by chemical, genetic or physical linking of the Ligand and the biological agent, or by mixing the above components, or by their co-administration.

The peptide, derivative or peptidomimetic of this invention has one or more of the following activities:

The present invention provides a pharmaceutical composition comprising at least one peptide or derivative thereof, wherein said polypeptide or derivative thereof is capable of specific binding with S100A4 wherein the polypeptide or derivative thereof comprises at least about 20% of the biological activity of the polypeptide or derivative thereof SEQ IDs. NO.: 5-15, 33-38.

The biological activity of the present pharmaceutical composition is measured but is not limited to using in vitro bioassays comprising Annexin 2 receptor binding assay, plasminogen processing assay or endothelial tube formation on Matrigel.RTM.

The invention further includes a pharmaceutical composition comprising one or more Ligands in association with a carrier. In another embodiment of the invention the Ligand is associated with a protein, polymer or any other carrier to improve the ability of the Ligand to interact with S100A4 or Annexin 2 and/or to improve pharmacological properties of the Ligand such as pharmacokinetics, stability and biodistribution. The association of the Ligand and the carrier can be achieved by chemical, genetic or physical linking of the Ligand and the carrier, or by mixing the above components, or by their co-administration.

In another yet embodiment of the invention the Ligand associated with a carrier is further associated with a biological agent which is achieved by chemical, genetic or physical linking of the Ligand—carrier composition described in the previous embodiment of the invention and a biological agent. The invention further provides a pharmaceutical composition comprising one or more Ligands or their complex with a carrier in association with a biological agent.

Moreover, the present invention extends to pharmaceutical compositions for diagnostics and/or treatment of diseases, where such diseases are associated with angiogenesis.

The invention is further directed to a pharmaceutical composition useful for diagnostics or treatment of angiogenesis associated diseases, comprising (a) any of the above peptides, variants or chemical derivatives including, but not limited to peptidomimetics and (b) a pharmaceutically acceptable carrier or excipient, either chemically conjugated or physically associated with a ligand.

The invention is further directed to a pharmaceutical composition useful for diagnostics or treatment of angiogenesis associated diseases, comprising any of the above peptides, variants or chemical derivatives including a peptidomimetic conjugated chemically or genetically fused to a therapeutic agent.

Also provided is a method for inhibition of S100A4-induced cell motility, metastasis formation, inflammation or angiogenesis in a subject having a disease or condition associated with undesired angiogenesis, comprising administering to the subject an effective amount of a pharmaceutical composition as described above.

In yet another embodiment of the invention the Ligand is associated with a modified biological agent, which is achieved by chemical linking of the Ligand to the modified biological agent, such as a pro-drug, many of which are known in the art, such as, for example, paclitaxel-PEG, and revealing no activity of the biological agent. The active biological agent is released from such an associate upon action of chemical or enzymatic reaction in the body.

The present invention also provides a method of treating a disease associated with angiogenesis in a patient in need of such therapy comprising administering to said patient an effective amount of said pharmaceutical composition of claim 1 and a pharmaceutical acceptable carrier.

In any of the foregoing methods, the disease or condition being treated may be primary tumor growth, tumor invasion or metastasis, atherosclerosis, post-balloon angioplasty vascular restenosis, neointima formation following vascular trauma, vascular graft restenosis, fibrosis associated with a chronic inflammatory condition, lung fibrosis, chemotherapy-induced fibrosis, wound healing with scarring and fibrosis, psoriasis, deep venous thrombosis, macular degeneration or another disease or condition in which angiogenesis is involved.

These and other aspects of the present invention will be better appreciated by reference to the following Sequence Listings and Detailed Description.

### DETAILED DESCRIPTION OF THE INVENTION

I. The Ligand

The present invention provides a pharmaceutical composition comprising at least one peptide, or derivatives thereof, which is capable of specific binding with S100A4 or Annexin 2, such a peptide or its derivative referenced herein as a “Ligand”. Since the Ligand is capable of specific binding with the receptors, it is also able to modulate S100A4 mediated angiogenesis in endothelial cells and tissues. Therefore, the present invention also provides pharmaceutical compositions in which the Ligand is used as a targeting moiety to improve the delivery of a biological agent used for therapeutic or diagnostic purposes.

In one preferred embodiment, the invention provides a Ligand of S100A4 or Annexin 2 of said ligand being peptide, or derivative thereof. The preparation of the peptides or derivatives thereof according to the invention is effected by means of one of the known organic chemical methods for peptide synthesis or with the aid of recombinant DNA techniques known.

II. Chemical Peptide Synthesis

The ligands of the present invention can be produced by means of chemical synthesis of peptides. There are numerous methods of chemical synthesis of peptides, that are known to those skilled in the art. These methods are for example described in: Methods of Organic Chemistry (Houben-Weyl), Synthesis of Peptides and Peptidomimetics—Workbench Edition Volume E22a and E22b (Editor-in-Chief Goodman M.) 2004 (Georg Thieme Verlag Stuttgart, New York), and in: The Peptides, Analysis, Synthesis, Biology Vol. 1-3 (Ed. Gross, E. and Meienhofer, J.) 1979, 1980, 1981 (Academic Press, Inc.). These methods have also been previously described in U.S. Pat. No. 6,733,755 B2, and in U.S. Pat. No. 6,696,274 B2, each of which is incorporated herein by reference. The methods of chemical synthesis of peptides include for example: stepwise synthesis of peptide chain by adding single amino acid residues, and conjugation of a peptide from shorter peptide fragments; these syntheses can be executed in solution or on the surface of insoluble support, and are usually multi-step processes. The respective methods further include the use of appropriate protection attached to chemical groups that are not intended to react during particular steps of the synthesis, and removing those protections when they are not longer needed. These methods include further removal of all protections, including the connection to the insoluble support if applicable, and purification of the product.

III. Biosynthetically Made Peptide

The ligands of the present invention can be prepared by recombinant methods, which are known to those skilled in the art. There are numerous bisynthetic methods of the production of peptides and proteins, and those methods have been described for example in laboratory manuals such as “Molecular Cloning: A Laboratory Manual”, Second Edition by Sambrook et al., Cold Spring Harbor Press, 1989; “Current Protocols in Molecular Biology”, Volumes I-III, Ausubel, R. M., ed., 1994; “Cell Biology: A Laboratory Handbook”, Volumes I-III, J. E. Celis, ed., 1994; “Current Protocols in Immunology”, Volumes I-III, Coligan, J. E., ed., 1994; “Oligonucleotide Synthesis”, M. J. Gait ed., 1984; “Nucleic Acid Hybridization”, B. D. Hames & S. J. Higgins eds., 1985; “Transcription And Translation”, B. D. Hames & S. J. Higgins, eds., 1984; “Animal Cell Culture”, R. 1. Freshney, ed., 1986; “Immunobilized Cells And Enzymes”, IRL Press, 1986; B. Perbal, “A Practical Guide To Molecular Cloning”, 1984. These methods have also been previously described in U.S. Pat. No. 6,733,755 B2, and in U.S. Pat. No. 6,696,274 B2, each of which is incorporated herein by reference.

One example of a method of producing the ligand using recombinant DNA techniques entails the steps of (1) generating DNA encoding product sequence, appropriated linkers and restriction sites coding sequences (2) inserting the DNA into an appropriate vector such as an expression vector, (3) inserting the gene containing vector into a microorganism or other expression system capable of expressing the gene, and (4) isolating and purifying the product. The presented invention may utilize one of many expression systems know to those skilled in the art. For example, the expression of the gene can be done in selected prokaryotic host cells, or in selected eukaryotic host cells, or in cell free systems. For each of these systems appropriate nucleotide sequences encoding the transcriptional and translational regulatory information need to be linked to the nucleotide sequence encoding the ligand, and appropriate procedures needs to be applied for introduction of the DNA construct into the system, and to produce the ligand, where such sequences and procedures are known to those skilled in the art.

In one preferred embodiment the ligand is biosynthetically produced by the expression system and is being isolated from this system.

In another embodiment, the peptide of present invention can be expressed as fusion proteins in which the ligands of invention are fused at its N-terminus or its C-terminus, or at both termini, to a polypeptide chain. In a preferred embodiment, the fusion protein is specifically cleavable such that at least a substantial portion of the peptide sequence can be proteolytically cleaved away from the fusion protein to yield the desired ligand.

In another embodiment, the fusion protein containing the ligand linked with another protein is further being used as one chemical entity. For example, the ligand can be obtained as a fusion protein with human IgG Fc fragment.

In another embodiment the ligand of present invention can be synthesized as a fusion protein with a virus coat protein and expressed on the surface of virus particle, for example bacteriophage M13, T7, T4 and lambda, lambda gt10, lambda gt11 and the like; adenovirus, retrovirus and pMAM-neo, pKRC and the like.

IV. Derivatives of Peptide

As used herein, the term “amino acid” and any reference to a specific amino acid is meant to include naturally occurring proteogenic amino acids as well as non-naturally occurring amino acids such as amino acid analogs. Those skilled in the art would know that this definition includes, unless otherwise specifically indicated, naturally occurring proteogenic (L)-amino acids, their optical (D)-isomers, chemically modified amino acids, including amino acid analogs such as penicillamine (3-mercapto-D-valine), naturally occurring non-proteogenic amino acids such as norleucine and chemically synthesized compounds that have properties known in the art to be characteristic of an amino acid. As used herein, the term “proteogenic” indicates that the amino acid can be incorporated into a protein in a cell through well-known metabolic pathways.

As used herein, the term “amino acid equivalent” refers to compounds, which depart from the structure of the naturally occurring amino acids, but which have substantially the structure of an amino acid, such that they can be substituted within a peptide, which retains is biological activity. Thus, for example, amino acid equivalents can include amino acids having side chain modifications or substitutions, and also include related organic acids, amides or the like. The term “amino acid” is intended to include amino acid equivalents. The term “residues” refers both to amino acids and amino acid equivalents.

As used herein, the term “peptide” is used in its broadest sense to refer to compounds containing amino acid equivalents or other non-amino groups, while still retaining the desired functional activity of a peptide. Peptide equivalents can differ from conventional peptides by the replacement of one or more amino acids with related organic acids (such as PABA), amino acids or the like or the substitution or modification of side chains or functional groups.

It is to be understood that limited modifications can be made to a peptide without destroying its biological function. Thus, modification of the peptides of the present invention that do not completely destroy their activity is within the definition of the compound claims as such. Modifications can include, for example, additions, deletions, or substitutions of amino acids residues, substitutions with compounds that mimic amino acid structure or functions, as well as the addition of chemical moieties such as amino or acetyl groups. The modifications can be deliberate or accidental, and can be modifications of the composition or the structure.

As used herein, “binding” refers to the ability of a given peptide to interact with a receptor such that the interaction between the peptide and the receptor is relatively specific. As used herein, the term “relatively specific” means that the affinity of binding of the receptor and peptide is about 1×10−5M or less. Therefore, the term “binding” does not encompass non-specific binding, such as non-specific adsorption to a surface. Non-specific binding can be readily identified by including the appropriate controls in a binding assay. Methods for determining the binding affinity are described in the Examples below.

The choice of including an (L)- or a (D)-amino acid into a peptide of the present invention depends, in part, on the desired characteristics of the peptide. For example, the incorporation of one or more (D)-amino acids can confer increasing stability on the peptide in vitro or in vivo. The incorporation of one or more (D)-amino acids also can increase or decrease the binding activity of the peptide as determined, for example, using the binding assays described herein, or other methods well known in the art. Replacement of all or part of a sequence of (L)-amino acids by the respective sequence of enantiomeric (D)-amino acids renders an optically isomeric structure in the respective part of the polypeptide chain. Inversion of the sequence of all or part of a sequence of (L)-amino acids renders a retro-analogues of the peptide. Combination of the enantiomeric (L to D, or D to L) replacement and inversion of the sequence renders retro-inverso-analogues of the peptide. It is known to those skilled in the art that enantiomeric peptides, their retro-analogues, and their retro-inverso-analogues maintain significant topological relationship to the parent peptide, and especially high degree of resemblance is often obtained for the parent peptide and its retro-inverso-analogues. Those relationship and resemblance can be reflected in biochemical properties of the peptides, especially in binding of the respective peptides and analogs to a receptor protein. The synthesis and properties of retro-inverso-analogues of peptides have been discussed for example in Methods of Organic Chemistry (Houben-Weyl), Synthesis of Peptides and Peptidomimetics—Workbench Edition Volume E22c (Editor-in-Chief Goodman M.) 2004 (Georg Thieme Verlag Stuttgart, New York), and in reference cited therein.

Derivatives of the peptides of the present invention can be produced by the chemical synthesis or they can be made using recombinant nucleic acid molecule techniques. Modifications to a specific peptide may be deliberate, as through site-directed mutagenesis and amino acid substitution during biosynthesis, or may be accidental such as through mutations in hosts, which produce the peptide. Peptides including derivatives can be obtained using standard mutagenesis techniques such as those described in Sambrook et al., Molecular Cloning, Cold Spring Harbor Laboratory Press (1989). For example, Chapter 15 of Sambrook describes procedures for site-directed mutagenesis of cloned DNA.

Derivatives of the peptides of the present invention prepared by recombinant methods include, but not limited by modification occurring during or after translation, for example, by phosphorylation, glycosylation, crosslinking, acylation, proteolytic cleavage, linkage to a therapeutic protein, an antibody molecule, membrane molecule or other ligand (see Ferguson et al., 1988, Annu. Rev. Biochem. 57:285-320).

Derivatives of the peptides of the present invention also include, but not limit by amino acid alterations such as deletions, substitutions, additions, and amino acid modifications. A “deletion” refers to the absence of one or more amino acid residue(s) in the related peptide. An “addition” refers to the presence of one or more amino acid residue(s) in the related peptide. Additions and deletions to a peptide may be at the amino terminus, the carboxy terminus, and/or internal, can be produced by chemical synthesis or by mutation encoding DNA, and/or by peptide post-translation modification.

Amino acid “modification” refers to the alteration of a naturally occurring amino acid to produce a non-naturally occurring amino acid. Derivatives of the peptides of the present invention with non-natural amino acids can be created by chemical synthesis or by site-specific incorporation of unnatural amino acids into polypeptides during the biosynthesis, as described in Christopher J. Noren, Spencer J. Anthony-Cahill, Michael C. Griffith, Peter G. Schultz, 1989 Science, 244:182-188.

A “substitution” refers to the replacement of one or more amino acid residue(s) by another amino acid residue(s) in the peptide. Derivatives of the peptides of the present invention containing a substitution can be made by chemical synthesis or by mutations in the encoding DNA for biosynthesis, such that a particular codon is changed to a codon, which codes for a different amino acid. Such a mutation is generally made by making the fewest nucleotide changes possible. A substitution mutation of this sort can be made to change an amino acid in the resulting peptide in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting peptide. To some extent the following groups contain amino acids which are interchangeable: the basic amino acids lysine, arginine, and histidine; the acidic amino acids aspartic and glutamic acids; the neutral polar amino acids serine, threonine, cysteine, glutamine, asparagine and, to a lesser extent, methionine; the nonpolar aliphatic amino acids glycine, alanine, valine, isoleucine, and leucine (however, because of size, glycine and alanine are more closely related and valine, isoleucine and leucine are more closely related); and the aromatic amino acids phenylalanine, tryptophan, and tyrosine. In addition, although classified in different categories, alanine, glycine, and serine seem to be interchangeable to some extent, and cysteine additionally fits into this group, or may be classified with the polar neutral amino acids. Although proline is a nonpolar neutral amino acid, its replacement represents difficulties because of its effects on conformation. Thus, substitutions by or for proline are not preferred, except when the same or similar conformational results can be obtained. The conformation conferring properties of proline residues may be obtained if one or more of these is substituted by hydroxyproline (Hyp). Derivatives can contain different combinations of alterations including more than one alteration and different types of alterations.

The ability of the derivative to retain some activity can be measured using techniques described herein and/or using techniques known to those skilled in the art for measuring the S100A4 or Annexin 2 binding activity. “Derivatives” of the peptides of the present invention are functional equivalents having similar amino acid sequence and retaining, to some extent, the activities of SEQ ID NO.: 5-15 and 33-38. By “functional equivalent” is meant the derivative has an activity that can be substituted for the activity of SEQ ID NO.: 5-15 and 33-38. Preferred functional equivalents retain the full level of S100A4 or Annexin 2 binding activity as measured by assays known to these skilled in the art, and/or in the assays described herein. Preferred functional equivalents have activities that are within 1% to 10,000% of the activity of SEQ ID NO.: 5-15 and 33-38, more preferably between 10% to 1000%, and more preferably within 50% to 200%. Derivatives have at least 50% sequence similarity, preferably 70%, more preferably 90%, and even more preferably 95% sequence similarity to SEQ ID NO.: 5-15 and 33-38. “Sequence similarity” refers to “homology” observed between amino acid sequences in two different polypeptides, irrespective of polypeptide origin.

In another embodiment derivatives of the peptides of the present invention can be produced by linking more than one copy of the ligand, or those of modified ligand, to another chemical molecule which is a chemical linker for the ligands. The geometry of the linker is such that the distance between the attachment points of the individual ligands is between 5 and 100 Angstrom. Such multiple copies of the ligand provide for an opportunity of multiple interactions of the respective derivative with the receptor molecule, or molecules, where the binding sites on the receptor molecules are typically separated by a distance between approximately 5 Angstroms and approximately 100 Angstroms. This is typically the distance of between approximately 5 to 100 atoms, such as C, N, O, etc., when they are connected linearly and unfolded. An embodiment provides that the chemical linker may comprise a linear chain of 18 atoms. A further embodiment provides that the chemical linker may comprise amino acids which may also comprise a linear chain of 18 atoms. Thus the derivative bearing multiple copies of the ligand has potentially increased the affinity to the receptor. Various suitable chemical linkers and methods of their production are known to those skilled in the art. The useful chemical linkers include, without limitation, a chemical molecule of molecular weight less than 1000D, an oligopeptide (less than 20 amino acid residues), a polypeptide (20 or more amino acid residues), a protein, a polymer, and other linker compounds known in the art, such as those provided in the examples. The respective derivatives can be produced either by chemical synthesis, or by biosynthesis.

The peptides of the present invention are as well useful when they are maintained in a constrained secondary conformation. As used herein, the terms “constrained secondary structure,” “stabilized” and “conformationally stabilized” indicate that the peptide bonds comprising the peptide are not able to rotate freely but instead are maintained in a relatively fixed structure.

Various methods for constraining the secondary structure of a peptide are well known in the art. For example, a peptide can be conformationally stabilized by incorporating it into it a sequence that forms particular secondary structure, such as a beta-turn or a helix, according to methods described, for example, by Dedhar et al., (1987) J. Cell. Biol. 104:585; by Rhodes et al., (1978) Biochem 17:3442; and by Carbone et al., (1987) J. Immunol 138:1838, each of which is incorporated herein by reference. Additionally, the peptides can be incorporated into larger linear, cyclic or branched peptides, so long as their receptor-binding activity is retained. The peptides of the present invention may be of any size so long as the S100A4 or Annexin 2 receptor-binding activity is retained, however, in one embodiment, peptides having twenty or fewer total amino acids are preferred.

A preferred method for constraining the secondary structure of a newly synthesized linear peptide is to cyclize the peptide using any of various methods well known in the art. For example, a cyclized peptide of the present invention can be prepared by forming a peptide bond between non-adjacent amino acid residues as described, for example, by Schiller et al., (1985) Int. J. Pept. Prot. Res. 25:171, which is incorporated herein by reference.

A newly synthesized linear peptide can also be cyclized by the formation of a bond between reactive amino acid side chains, such as for example cysteine residues forming a disulfide bridge, or acidic amino acids and lysine forming lactame. Methods for forming these and other bonds are well known in the art and are based on well-known rules of chemical reactivity (Morrison and Boyd, Organic Chemistry, 6th Ed. (Prentice Hall, 1992), which is herein incorporated by reference).

V. Peptidomimetics

Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15: 29; Veber and Freidinger (1985) TINS p. 392; and Evans et al. (1987) J. Med. Chem 30: 1229, which are incorporated herein by reference) and can be developed, for example, with the aid of computerized molecular modeling. Synthesis of various types of peptidomimetics has been reviewed for example in: Methods of Organic Chemistry (Houben-Weyl), Synthesis of Peptides and Peptidomimetics—Workbench Edition Volume E22c (Editor-in-Chief Goodman M.) 2004 (Georg Thieme Verlag Stuttgart, New York).

In a preferred embodiment, the present invention provides a pharmaceutical composition comprising at least one polypeptide or derivative thereof, wherein the polypeptide or derivative thereof is capable of specific binding with S100A4 or Annexin 2 comprises peptidomimetics that are capable of specific binding with S100A4 or Annexin 2.

Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as SEQ ID NO.: 5-15 and 33-38, but have one or more peptide linkages optionally replaced by a linkage selected from the group consisting of: —CH2-NH—, —CH2S—, —CH2-CH.2-, —CH═CH— (cis and trans), —COCH2-, —CH(OH)CH2—, and —CH2SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends Pharm Sci (1980) pp. 463-468 (general review); Hudson, D. et al., (1979) Int J Pept Prot Re 14:177-185 (—CH2 NH—, —CH2-CH2-); Spatola, A. F. et al., (1986) Life Sci 38:1243-1249 (—CH2-S); Hann, M. M., (1982) J Chem Soc Perkin Trans 1307-314 (—CH═CH—, cis and trans); Almquist, R. G. et al., (1980) J Med Chem 23: 1392-1398 (—COCH2-); Jennings-White, C. et al., (1982) Tetrahedron Lett 23:2533 (—COCH2-); Szelke, M. et al., European Appin. EP 45665 (1982) CA: 97:39405 (1982) (—CH(OH)CH.2-); Holladay, M. W. et al., (1983) Tetrahedron Lett 24:4401-4404 (—C(OH)CH2-); and Hruby, V. J., (1982) Life Sci 31:189-199 (—CH2-S—); each of which is incorporated herein by reference.

In another embodiment, a particularly preferred non-peptide linkage is —CH.2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) (e.g., are not contact points in S100A4-Annexin 2 complexes) to which the peptidomimetic binds to produce the therapeutic effect. Derivitization (e.g., labelling) of peptidomimetics should not substantially interfere with the desired biological or pharmacological activity of the peptidomimetic.

A variety of designs for peptide mimetics are possible. For example, cyclic peptides, in which the necessary conformation for binding is stabilized by nonpeptides, are specifically contemplated. U.S. Pat. No. 5,192,746 to Lobl, et al., U.S. Pat. No. 5,169,862 to Burke, Jr., et al, U.S. Pat. No. 5,539,085 to Bischoff, et al., U.S. Pat. No. 5,576,423 to Aversa, et al., U.S. Pat. No. 5,051,448 to Shashoua, and U.S. Pat. No. 5,559,103 to Gaeta, et al., all hereby incorporated by reference, describe multiple methods for creating such compounds. Synthesis of nonpeptide compounds that mimic peptide sequences is also known in the art. Eldred, et al., (J. Med. Chem. 37:3882 (1994)) describe nonpeptide antagonists that mimic the peptide sequence. Likewise, Ku, et al., (J. Med. Chem. 38:9 (1995)) give further elucidation of the synthesis of a series of such compounds.

VI. Biological Agents

A variety of biological agents are suitable for use in the invention. These include, without limitation, proteins, peptides (e.g., oligopeptides and polypeptides) including cytokines, hormones (such as insulin), and the like, recombinant soluble receptors, monoclonal antibodies, human growth hormones, tissue plasminogen activators, clotting factors, vaccines, colony stimulating factors, erythropoietins, enzymes, and dismultase. Therefore, in on embodiment, the present pharmaceutical composition comprising at least one polypeptide or derivative thereof, wherein the polypeptide or derivative thereof is capable of specific binding with S100A4 or Annexin 2 further comprises a biological agent.

Preferred classes of biological agents (including chemotherapeutic agents) include anti-neoplastic agents, antibacterial agents, antiparasitic agents, anti-fungal agents, CNS agents, immunomodulators and cytokines, toxins and neuropeptides. Biological agents for which target cells tend to develop resistance mechanisms are also preferred. Particularly preferred biological agents include anthracyclines such as doxorubicin, daunorubicin, epirubicin, idarubicin, mithoxanthrone or carminomycin, vinca alkaloids, mitomycin-type antibiotics, bleomycin-type antibiotics, azole antifungals such as fluconazole, polyene antifungals such as amphotericin B, taxane-related antineoplastic agents such as paclitaxel and immunomodulators such as tumor necrosis factor alpha (TNF-alpha), interferons and cytokines.

Preferred biological agents (including chemotherapeutic agents) include without limitation additional antifungal agents such as amphotericin-B, flucytosine, ketoconazole, miconazole, itraconazole, griseofulvin, clotrimazole, econazole, terconazole, butoconazole, ciclopirox olamine, haloprogin, toinaftate, naftifine, nystatin, natamycin, undecylenic acid, benzoic acid, salicylic acid, propionic acid and caprylic acid. Such agents further include without limitation antiviral agents such as zidovudine, acyclovir, ganciclovir, vidarabine, idoxuridine, trifluridine, foxcarnet, amantadine, rimantadine and ribavirin. Such agents further include without limitation antibacterial agents such as penicillin-related compounds including 9-lactam antibiotics, broad spectrum penicillins and penicillinase-resistant penicillins (such as methicillin, nafcillin, oxacillin, cloxacillin, dicloxacillin, amoxicillin, ampicillin, ampicillin-sulbactam, azocillin, bacampicillin, carbenicillin, carbenicillin indanyl, cyclacillin, mezlocillin, penicillin G, penicillin V, piperacillin, ticarcillin, imipenem and aztreonam), cephalosporins (cephalosporins include first generation cephalosporins such as cephapirin, cefaxolin, cephalexin, cephradine and cefadroxil; second generation cephalosporins such as cefamandole, cefoxitin, cefaclor, cefuroxime, cefuroxime axetil, cefonicid, cefotetan and ceforamide; third generation cephalosporins such as cefotaxime, ceftizoxime, ceftriaxone, cefoperazone and ceftazidime), tetracyclines (such as demeclocytetracycline, doxycycline, methacycline, minocycline and oxytetracycline), beta-lactamase inhibitors (such as clavulanic acid), aminoglycosides (such as amikacin, gentamicin C, kanamycin A, neomycin B, netilmicin, streptomycin and tobramycin), chloramphenicol, erythromycin, clindamycin, spectinomycin, vancomycin, bacitracin, isoniazid, rifampin, ethambutol, aminosalicylic acid, pyrazinamide, ethionamide, cycloserine, dapsone, sulfoxone sodium, clofazimine, sulfonamides (such as sulfanilamide, sulfamethoxazole, sulfacetamide, sulfadiazine, and sulfisoxazole), trimethoprim-sulfamethoxazole, quinolones (such as nalidixic acid, cinoxacin, norfloxacin and ciprofloxacin), methenamine, nitrofurantoin and phenazopyridine. Such agents further include agents active against protozoal infections such as chloroquine, diloxamide furoate, emetine or dehydroemetine, 8-hydroxyquinolines, metronidazole, quinacrine, melarsoprol, nifurtimox, pentamidine, sodium stibogluconate and suramin.

A variety of central nervous system agents are suitable for use in the present composition. These include neuroleptics such as the phenothiazines (such as compazine, thorazine, promazine, chlorpromazine, acepromazine, aminopromazine, perazine, prochlorperazine, trifluoperazine, and thioproperazine), rauwolfia alkaloids (such as reserpine and deserpine), thioxanthenes (such as chlorprothixene and tiotixene), butyrophenones (such as haloperidol, moperone, trifluoperidol, timiperone, and droperidol), diphenylbutylpiperidines (such as pimozide), and benzamides (such as sulpiride and tiapride); tranquilizers such as glycerol derivatives (such as mephenesin and methocarbamol), propanediols (such as meprobamate), diphenylmethane derivatives (such as orphenadrine, benzotrapine, and hydroxyzine), and benzodiazepines (such as chlordiazepoxide and diazpam); hypnotics (such as zolpdem and butoctamide); 9-blockers (such as propranolol, acebutonol, metoprolol, and pindolol); antidepressants such as dibenzazepines (such as imipramine), dibenzocycloheptenes (such as amitriptyline), and the tetracyclics (such as mianserine); MAO inhibitors (such as pheneizine, iproniazide, and selegeline); psychostimulants such as phenylethylamine derivatives (such as amphetamines, dexamphetamines, fenproporex, phentermine, amfepramone, and pemline) and dimethylaminoethanols (such as clofenciclan, cyprodenate, a minorex, and mazindol); GABA-mimetics (such as progabide), alkaloids (such as co-dergocrine, dihydroergocristine, and vincamine); cholinergics (such as citicoline and physosigmine); vasodilators (such as pentoxifyline); and cerebro active agents (such as pyritinol and meclofenoxate); as well as mixtures of several such agents.

Of particular interest are sedative-hypnotics such as the benzodiazepines, psycho-pharmacological agents such as the phenothiazines, thioxanthenes, butyrophenones, and dibenzoxazepines, and central nervous system stimulants. In one embodiment, the pharmaceutical composition of the present invention further comprises a biological agent that can be applied to a wide variety of central nervous system agents by applying the principles and procedures described herein.

The compositions also can utilize a variety of polypeptides such as antibodies, toxins such as diphtheria toxin, peptide hormones, such as colony stimulating factor, and tumor necrosis factors, neuropeptides, growth hormone, erythropoietin, and thy-roid hormone, lipopro-teins such as alpha-lipoprotein, proteoglycans such as hyaluronic acid, glycoproteins such as gonado-tropin hormone, immunomodulators or cytokines such as the interferons or interleukins, hormone receptors such as the estrogen recep-tor. Preferred peptides are those with molecular weight of at least about 1,000, more preferably at least about 5,000, most preferably at least about 10,000. The compositions also can be utilize enzyme inhibiting agents such as reverse transcriptase inhibitors, protease inhibitors, angiotensin converting enzymes, 5alpha-reductase, and the like. Typical of these agents are peptide and nonpeptide structures such as finasteride, quinapril, ramipril, lisinopril, saquinavir, ritonavir, indinavir, nelfinavir, zidovudine, zalcitabine, allophenylnorstatine, kynostatin, dela-viridine, bis-tetrahydrofuran ligands (see, for example Ghosh et al., J. Med. Chem. 39 (17): 3278-90 1966), and didanosine. Such agents can be administered alone or in combination therapy; e.g., a combination therapy utilizing saquinavir, zalcitabine, and didanosine or saquinavir, zalcitabine, and zidovudine. See, for example, Collier et al., Antiviral Res., 1996 Jam. 29 (1): 99. A variety of human and animal cytokines are suitable for use in the present compositions. These include interferons, interleukins, tumor necrosis factors (TNFs) such as TNFalpha, and a number of other protein and peptide factors controlling functions of the immune system. It will be appreciated that this extends to mixtures of several such agents, and the invention is not directed to the underlying specific activity of the cytokines themselves, but rather to the compositions themselves.

VII. Carriers

A variety of carriers can be associated with the ligand including, but not limiting by synthetic, semi-synthetic and natural compounds such as polypeptides, lipids, carbohydrates, polyamines, synthetic polymers, that form solutions (unimolecular systems), dispersions (supramolecular systems), or any particular systems such as nanoparticles, microspheres, matrixes, gels and other. The applicable carriers have previously been described in U.S. Pat. No. 6,733,755 B2, and in U.S. Pat. No. 6,696,274 B2.

In one embodiment, this invention provides a pharmaceutical composition comprising at least one polypeptide or derivative thereof, wherein said polypeptide or derivative thereof is capable of specific binding with S100A4 or Annexin 2 further comprises a carrier. The invention further provides a pharmaceutical composition comprising one or more Ligands or their complex with a carrier in association with a biological agent

The following classes of carriers are given as examples. It is understood, however, that a variety of other carriers can be used in the present invention.

The polymeric carriers can be nonionic water-soluble, nonionic hydrophobic or poorly water soluble, cationic, anionic or polyampholite, such as a polypeptides. It is preferred that the degrees of polymerization of these polymer carriers were from about 3 to about 500,000 more preferably from about 5 to about 5000, still more preferably from about 20 to about 500.

Preferred hydrophilic carrier is a nontoxic and non-immunogenic polymer which is soluble in water. Such segments include (but not are limited to) polyethers (e.g., polyethylene oxide), polysaccharides (e.g., dextran), polyglycerol, homopolymers and copolymers of vinyl monomers (e.g., polyacrylamide, polyacrylic esters (e.g., polyacryloyl morpholine), polymethacrylamide, poly(N-(2-hydroxypropyl)methacrylamide, polyvinyl alcohol, polyvinyl pyrrolidone, polyvinyltriazole, N-oxide of polyvinylpyridine, copolymer of vinylpyridine and vinylpyridine N-oxide) polyortho esters, polyaminoacids, polyglycerols (e.g., poly-2-methyl-2-oxazoline, poly-2-ethyl-2-oxazoline) and copolymers and derivatives thereof.

Preferred nonionic hydrophobic and poorly water soluble segments include polypropylene oxide, copolymers of polyethylene oxide and polyethylene oxide, polyalkylene oxide other than polyethylene oxide and polypropylene oxide, homopolymers and copolymers of styrene (e.g., polystyrene), homopolymers and copolymers isoprene (e.g., polyisoprene), homopolymers and copolymers butadiene (e.g., polybutadiene), homopolymers and copolymers propylene (e.g., polypropylene), homopolymers and copolymers ethylene (e.g., polyethylene), homopolymers and copolymers of hydrophobic aminoacids and derivatives of aminoacids (e.g., alanine, valine, isoleucine, leucine, norleucine, phenylalanine, tyrosine, tryptophan, threonine, proline, cistein, methionone, serine, glutamine, aparagine), homopolymers and copolymers of nucleic acid and derivatives thereof.

Preferred polyanionic carrier include those such as polymethacrylic acid and its salts, polyacrylic acid and its salts, copolymers of methacrylic acid and its salts, copolymers of acrylic acid and its salts, heparin, polyphosphate, homopolymers and copolymers of anionic aminoacids (e.g., glutamic acid, aspartic acid), polymalic acid, polylactic acid, polynucleotides, carboxylated dextran, and the like.

Preferred polycationic carrier include polylysine, polyasparagine, homopolymers and copolymers of cationic aminoacids (e.g., lysine, arginine, histidine), alkanolamine esters of polymethacrylic acid (e.g., poly-(dimethylammonioethyl methacrylate), polyamines (e.g., spermine, polyspermine, polyethyleneimine, polypropyleneimine, polybutileneimine, poolypentyleneimine, polyhexyleneimine and copolymers thereof), copolymers of tertiary amines and secondary amines, partially or completely quaternized amines, polyvinyl pyridine and the quaternary ammonium salts of the polycation segments. These preferred polycation segments also include aliphatic, heterocyclic or aromatic ionenes (Rembaum et al., Polymer letters, 1968, 6; 159; Tsutsui, T., In Development in ionic polymers-2, Wilson A. D. and Prosser, H. J. (eds.) Applied Science Publishers, London, new York, vol. 2, pp. 167-187, 1986).

Additionally, dendrimers, for example, polyamidoamines of various generations (Tomalia et al., Angew. Chem., Int. Ed. Engl. 1990, 29, 138) can be also used.

Particularly preferred are copolymers selected from the following polymer groups:
(a) segmented copolymers having at least one hydrophilic nonionic polymer and at least one hydrophobic nonionic segment (“hydrophilic-hydrophobic copolymers”);
(b) segmented copolymers having at least one cationic segment and at least one nonionic segment (“cationic copolymers”);
(c) segmented copolymers having at least one anionic segment and at least one nonionic segment (“anionic copolymers”);
(d) segmented copolymers having at least one polypetide segment and at least one non-peptide segment (“polypeptide copolymers”);
(e) segmented copolymers having at least one polynucleotide segment and at least one segment which is not a nucleic acid “polypeptide copolymers”);

In one preferred embodiment, the segmented copolymers are the block copolymers of ethylene oxide and propylene oxide.

VIII. Associating Biological Agents and Carriers with the Ligand

A. Conjugation of the Ligand

In another preferred embodiment the invention provides a Ligand of S100A4 or Annexin 2 conjugated to a therapeutic agent. Preferred therapeutic agents are described in the above description of invention.

In yet another preferred embodiment the invention provides a Ligand of S100A4 Annexin 2 or derivative of the Ligand, conjugated to a drug carrier system, such a carrier system being a polymer molecule, a block copolymer molecule, or a derivative of said polymer. The carrier system may also comprise a protein molecule. Preferred carrier systems are described in the above description of invention.

B. Chemical Conjugation of the Ligand to the Therapeutic Agent or a Carrier

The preparation of the conjugates of the Ligand to the therapeutic agent, or to the carrier system is effected by means of one of the known organic chemical methods for chemical ligation. The structural link between the Ligand and the macromolecule, as well as the chemical method by which they are joined, should be chosen so that the binding ability of the Ligand and the biological activity of the Ligand, when joined in the conjugate, are minimally compromised. As will be appreciated by those skilled in the art, there are a number of suitable chemical conjugation methods. The selection of the appropriate conjugation method can be rationalized by the inspection of the chemical groups present in the conjugated molecules, as well as evaluation of possible modification of these molecules to introduce some new chemical groups into them. Numerous chemical groups can subject conjugation reactions. The following groups are mentioned here as examples: hydroxyl group (—OH), primary and secondary amino group (—NH2 and —NH—), carboxylic group (—COOH), sulfhydryl group (—SH), aromatic rings, sugar residues, aldehydes (—CHO), alphatic and aromatic halides, and others. Reactivity of these groups is well known in the art (Morrison and Boyd, Organic Chemistry, 6th Ed. (Prentice Hall, 1992), Jerry March, Advanced Organic Chemistry, 4th Ed. (Wiley 1992), which are herein incorporated by reference). A more extensive description of conjugation methods and techniques can be found in: G. T. Harmanson, Bioconjugate Techniques, Academic Press, Inc. 1995, and in: S. S. Wong, Chemistry of Protein Conjugation and Cross-Linking, CRC Press, Inc. 1991, which are herein incorporated by reference.

C. Genetical Fusion of Ligand with Therapeutic Polypeptide

In another embodiment of the invention, natural, modified, or recombinant nucleic acid sequences encoding the Ligand may be ligated to a biological active polypeptide sequence to encode a fusion protein. The general methods suitable for the construction and expression of Ligand fusions with therapeutic proteins are the same as those described herein above for recombinant production of Ligand. Chimeric Ligand-polypeptides may be most conveniently constructed by fusing in-frame the DNA sequence encoding the Ligand of present invention to a cDNA sequence encoding the polypeptide of interest. However, fusion to genomic fragments of therapeutic polypeptides can also be used. Alternatively, PCR techniques can be used to join the two parts of the molecule in-frame with an appropriate vector. Spacer of various length and structure can be inserted between the Ligand sequence and therapeutic protein in order to provide the fusion protein with additional flexibility and preserve the protein folding. The fusions of Ligand of the present invention can be purified by various well-known methods including affinity chromatography and immobilized metal chelate chromatography (Al-Mashikhi et al., J. Dairy Sci. 71:1756-1763 [1988]). Suitable fusion partners are as discussed in Section “Biological agents”.

D. Introduction of Ligand in Virus Proteins

The Ligand of present invention can be introduced into viral particles in order to change a tropism of virus. Different viruses are capable of being used as vectors for the in vivo transfer and expression of genes. By way of example, retroviruses (RSV, HMS, MMS, and the like), HSV virus, adeno-associated viruses, adenoviruses, vaccinia virus, and the like, may be mentioned.

For example, the targeting of adenoviruses can be provided by construction of chimeric adenovirus fiber protein, which differs from the wild-type coat protein by the introduction of the Ligand amino acid sequence in a conformationally-restrained manner. Such a chimeric adenovirus fiber protein will be able to direct entry into cells of a vector comprising the chimeric fiber protein that is more efficient than entry into cells of a vector that is identical except for comprising a wild-type adenovirus fiber protein rather than the chimeric adenovirus fiber protein.

Desirably, the Ligand encoding sequence is introduced into the fiber protein at the level of gene expression. Such the Ligand amino acid sequence either is introduced in place of adenoviral sequences, or in addition to adenoviral sequences. Regardless of the nature of the introduction, its integration into an adenoviral fiber protein at the level of either DNA or protein, results in the generation of a chimeric fiber protein containing peptide motif of the peptide of the present invention.

Redirecting viral vectors to S100A4 or Annexin 2 expressing tissues can be achieved by using bispesific conjugates produced by chemical linkage of anti-virus antibody to the Ligand of present invention, as described, for example for anti-adenovirus antibody (Haisma H J., et al., Cancer Gene Ther., 2000, Alvarez R D., et al., Clin. Cancer Res., 2000). To avoid the limitation of chemical conjugations, genetically fused proteins comprising of anti-fiber knob AB (or cellular adenovirus receptor, CAR) and the Ligand can be produced (Dmitriev I et al., J. Virol., 2000).

The following examples are intended merely to illustrate the best mode now known for practicing the invention but the invention is not to be considered as limited to the details of such examples.

TABLE 1
List of polypeptide sequences

| | |
|---|---|
| SEQ ID 5 | LREVY-GASLAS-YMERW |
| SEQ ID 6 | Ac-AGLREVYGASLASYMERW-amide |

SEQ ID 6 is SEQ ID 5 supplemented with blocking groups required for
experiments described in Examples 10 and 12.

| | |
|---|---|
| SEQ ID 7 | Fam-AGLREVYGASLASYMERW-amide |

SEQ ID 7 is SEQ ID 5 supplemented with blocking groups and a fluorescent tag
required for experiments described in Examples 8 and 9.

| | |
|---|---|
| SEQ ID 8 | Fam-CAGLREVYGASLASYMERW-amide |

SEQ ID 8 is SEQ ID 5 supplemented with blocking groups, reactive SH group,
and a fluorescent tag required for experiments described in Examples 14, 15, 16 and 17.

| | |
|---|---|
| SEQ ID 9 | Fam-aGlrevyGASlASymerw-amide |

SEQ ID 9 is a derivative of SEQ ID 37 supplemented with blocking groups and a
fluorescent tag required for experiments described in Examples 8 and 9.

| | |
|---|---|
| SEQ ID 10 | Fam-aGwremysalsASyverl-amide |

SEQ ID 10 is SEQ ID 37 supplemented with blocking groups and a fluorescent
tag required for experiments described in Examples 8 and 9.

| | |
|---|---|
| SEQ ID 11 | AGLREVYGASLASYMERW-amide |

SEQ ID 11 is SEQ ID 5 supplemented with a blocking group and a reactive NH₂
group required for experiments described in Examples 18, 19, and 20.

| | |
|---|---|
| SEQ ID 12 | YMERW |
| SEQ ID 13 | Fam-ASYMERW-amide |

SEQ ID 13 is SEQ ID 12 supplemented with blocking groups and a fluorescent
tag required for experiments described in Example 8.

| | |
|---|---|
| SEQ ID 14 | LREVY |
| SEQ ID 15 | Fam-AGLREVY-amide |

SEQ ID 15 is SEQ ID 14 supplemented with blocking groups and a fluorescent
tag required for experiments described in Example 8.

SEQ ID 28

MACPLEKALDVMVSTFHKYSGKEGDKFKLNKSELKELLTRELPSFLGKRTDEAA
FQKLMSNLDSNRDNEVDFQEYCVFLSCIAMMCNEFFEGFPDKQPRKK

SEQ ID 29

STVHEILCKLSLEGDHSTPPSAYGSVKAYTNFDAERDALNIETAIKTKGVDEVTIV
NILTNRSNAQRQDIAFAYQRRTKKELASALKSALSGHLETLILGLLKTPAQYDASE
LKASMKGLGTDEDSLIEIICSRTNQELQEINRVYKEMYKTDLEKDIISDTSGDFRK
LMVALAKGRRAEDGSVIDYELIDQDARDLYDAGVKRKGTDVPKWISIMTERSVP
HLQKVFDRYKSYSPYDMLESIRKEVKGDLENAFLNLVQCIQNKPLYFADRLYDS
MKGKGTRDKVLIRIMVSRSEVDMLKIRSEFKRKYGKSLYYYIQQDTKGDYQKAL
LYLCGGDD

SEQ ID 30

HSTPPSAYGSVKAYTNFDAERDALNIETAIKTKGVDEVTIVNILTNRSNAQRQD
IAFAYQRRTKKELASALKSALSGHLETLILGLLKTPAQYDASELKASMKGLGTDE
DSLIEIICSRTNQELQEINRVYKEMYKTDLEKDIISDTSGDFRKLMVALAKGRRAE
DGSVIDYELIDQDARDLYDAGVKRKGTDVPKWISIMTERSVPHLQKVFDRYKSY
SPYDMLESIRKEVKGDLENAFLNLVQCIQNKPLYFADRLYDSMKGKGTRDKVLI
RIMVSRSEVDMLKIRSEFKRKYGKSLYYYIQQDTKGDYQKALLYLCGGDD

SEQ ID 31

VKAYTNFDAERDALNIETAIKTKGVDEVTIVNILTNRSNAQRQDIAFAYQRRTKK
ELASALKSALSGHLETLILGLLKTPAQYDASELKASMKGLGTDEDSLIEIICSRTNQ
ELQEINRVYKEMYKTDLEKDIISDTSGDFRKLMVALAKGRRAEDGSVIDYELIDQ
DARDLYDAGVKRKGTDVPKWISIMTERSVPHLQKVFDRYKSYSPYDMLESIRKE
VKGDLENAFLNLVQCIQNKPLYFADRLYDSMKGKGTRDKVLIRIMVSRSEVDML
KIRSEFKRKYGKSLYYYIQQDTKGDYQKALLYLCGGDD

SEQ ID 32

MPSQMEHAMETMMFTFHKFAGDKGYLTKEDLRVLMEKEFPGFLENQKDPLAV
DKIMKDLDQCRDGKVGFQSFFSLIAGLTIACNDYFVVHMKQKGKK

| | |
|---|---|
| SEQ ID 33 | ymerw |
| SEQ ID 34 | Irevy |
| SEQ ID 35 | wremy |
| SEQ ID 36 | ywerl |
| SEQ ID 37 | wremysalsASyverl |
| SEQ ID 38 | Ac-aGwremysalsASyverl-amide |

SEQ ID 38 is SEQ ID 37 supplemented with blocking groups required for
experiments described in Example 12.

SEQ ID 39

MPLLLLLPLLWAGALAMDKLASGTLEDISRPVCWAAADPGLREVYGAGLAGYM
ERWASGGDPIEGRGGGGGDPKSCDKPHTCPLCPAPELLGGPSVFLFPPKPKDTLM
ISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPS
RDELTKNQVSLTCLVKGFYPSDIAVEWESNGQPENNYKATPPVLDSDGSFFLYSK
LTVDKSRWQQGNVFSCSVMHEALHNHYTQKSLSLSPGK

| | |
|---|---|
| SEQ ID 41 | YMERV |
| SEQ ID 42 | YMERL |
| SEQ ID 43 | YMERI |
| SEQ ID 44 | YMERF |
| SEQ ID 45 | YMERH |
| SEQ ID 46 | YVERW |
| SEQ ID 47 | YVERV |
| SEQ ID 48 | YVERL |
| SEQ ID 49 | YVERI |
| SEQ ID 50 | YVERE |
| SEQ ID 51 | YVERH |
| SEQ ID 52 | YLERW |
| SEQ ID 53 | YLERV |
| SEQ ID 54 | YLERL |
| SEQ ID 55 | YLERI |
| SEQ ID 56 | YLERF |
| SEQ ID 57 | YLERH |
| SEQ ID 58 | YIERW |
| SEQ ID 59 | YIERV |
| SEQ ID 60 | YIERL |
| SEQ ID 61 | YIERI |
| SEQ ID 62 | YIERF |
| SEQ ID 63 | YIERH |
| SEQ ID 64 | FMERW |
| SEQ ID 65 | FMERV |
| SEQ ID 66 | FMERL |
| SEQ ID 67 | FMERI |
| SEQ ID 68 | FMERF |
| SEQ ID 69 | FMERH |
| SEQ ID 70 | FVERW |
| SEQ ID 71 | FVERV |
| SEQ ID 72 | FVERL |
| SEQ ID 73 | FVERI |
| SEQ ID 74 | FVERF |
| SEQ ID 75 | FVERH |
| SEQ ID 76 | FLERW |
| SEQ ID 77 | FLERV |
| SEQ ID 78 | FLERL |
| SEQ ID 79 | FLERI |
| SEQ ID 80 | FLERF |
| SEQ ID 81 | FLERH |
| SEQ ID 82 | FIERW |
| SEQ ID 83 | FIERV |
| SEQ ID 84 | FIERL |
| SEQ ID 85 | FIERI |
| SEQ ID 86 | FIERF |
| SEQ ID 87 | FIERH |

SEQ ID 88

| | |
|---|---|
| 1 | ATGCCGCTGC TGCTACTGCT GCCCCTGCTG TGGGCAGGGG CCCTGGCTAT |
| 51 | GGATAAGCTT GCTAGCGGTA CCCTCGAGGA TATCTCTAGA CCAGTGTGCT |
| 101 | GGGCGGCCGC GGATCCAGGA CTGCGCGAGG TGTACGGAGC GGGCCTTGCC |
| 151 | GGGTACATGG AGCGCTGGGC TAGCGGAGGG GATCCCATCG AAGGTCGTGG |
| 201 | TGGTGGTGGT GGTGATCCCA AATCTTGTGA CAAACCTCAC ACATGCCCAC |
| 251 | TGTGCCCAGC ACCTGAACTC CTGGGGGGAC CGTCAGTCTT CCTCTTCCCC |
| 301 | CCAAAACCCA AGGACACCCT CATGATCTCC CGGACCCCTG AGGTCACATG |
| 351 | CGTGGTGGTG GACGTGAGCC ACGAAGACCC TGAGGTCAAG TTCAACTGGT |
| 401 | ACGTGGACGG CGTGGAGGTG CATAATGCCA AGACAAAGCC GCGGGAGGAG |
| 451 | CAGTACAACA GCACGTACCG TGTGGTCAGC GTCCTCACCG TCCTGCACCA |
| 501 | GGACTGGCTG AATGGCAAGG AGTACAAGTG CAAGGTCTCC AACAAAGCCC |
| 551 | TCCCAGCCCC CATCGAGAAA ACCATCTCCA AAGCCAAAGG GCAGCCCCGA |
| 601 | GAACCACAGG TGTACACCCT GCCCCCATCC CGGGATGAGC TGACCAAGAA |
| 651 | CCAGGTCAGC CTGACCTGCC TAGTCAAAGG CTTCTATCCC AGCGACATCG |
| 701 | CCGTGGAGTG GGAGAGCAAT GGGCAGCCGG AGAACAACTA CAAGGCCACG |
| 751 | CCTCCCGTGC TGGACTCCGA CGGCTCCTTC TTCCTCTACA GCAAGCTCAC |
| 801 | CGTGGACAAG AGCAGGTGGC AGCAGGGGAA CGTCTTCTCA TGCTCCGTGA |
| 851 | TGCATGAGGC TCTGCACAAC CACTACACGC AGAAGAGCCT CTCCCTGTCT |
| 901 | CCGGGTAAAT GA |

SEQ ID 88 is the nucleotide sequence encoding SEQ ID 39. SEQ ID 88 uses the
common abbreviations for nucleotides.

SEQ ID 89

MPLLLLLPLLWAGALAMDKLASGTLEDISRPVCWAAADPGLREVYASGGDPIEG
RGGGGGDPKSCDKPHTCPLCPAPELLGGPSVFLFPPKPKDTLMISRTPEVTCVVV
DVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKG
FYPSDIAVEWESNGQPENNYKATPPVLDSDGSFFLYSKLTVDKSRWQQGNVFSCS
VMHEALHNHYTQKSLSLSPGK

SEQ ID 90

| | |
|---|---|
| 1 | ATGCCGCTGC TGCTACTGCT GCCCCTGCTG TGGGCAGGGG CCCTGGCTAT |
| 51 | GGATAAGCTT GCTAGCGGTA CCCTCGAGGA TATCTCTAGA CCAGTGTGCT |
| 101 | GGGCGGCCGC GGATCCAGGA CTGCGCGAGG TGTACGCTAG CGGAGGGGAT |
| 151 | CCCATCGAAG GTCGTGGTGG TGGTGGTGGT GATCCCAAAT CTTGTGACAA |
| 201 | ACCTCACACA TGCCCACTGT GCCCAGCACC TGAACTCCTG GGGGGACCGT |
| 251 | CAGTCTTCCT CTTCCCCCCA AAACCCAAGG ACACCCTCAT GATCTCCCGG |
| 301 | ACCCCTGAGG TCACATGCGT GGTGGTGGAC GTGAGCCACG AAGACCCTGA |
| 351 | GGTCAAGTTC AACTGGTACG TGGACGGCGT GGAGGTGCAT AATGCCAAGA |
| 401 | CAAAGCCGCG GGAGGAGCAG TACAACAGCA CGTACCGTGT GGTCAGCGTC |
| 451 | CTCACCGTCC TGCACCAGGA CTGGCTGAAT GGCAAGGAGT ACAAGTGCAA |
| 501 | GGTCTCCAAC AAAGCCCTCC CAGCCCCCAT CGAGAAAACC ATCTCCAAAG |
| 551 | CCAAAGGGCA GCCCCGAGAA CCACAGGTGT ACACCCTGCC CCCATCCCGG |
| 601 | GATGAGCTGA CCAAGAACCA GGTCAGCCTG ACCTGCCTAG TCAAAGGCTT |
| 651 | CTATCCCAGC GACATCGCCG TGGAGTGGGA GAGCAATGGG CAGCCGGACA |
| 701 | ACAACTACAA GGCCACGCCT CCCGTGCTGG ACTCCGACGG CTCCTTCTTC |
| 751 | CTCTACAGCA AGCTCACCGT GGACAAGAGC AGGTGGCAGC AGGGGAACGT |
| 801 | CTTCTCATGC TCCGTGATGC ATGAGGCTCT GCACAACCAC TACACGCAGA |
| 851 | AGAGCCTCTC CCTGTCTCCG GGTAAATGA |

SEQ ID 90 is the nucleotide sequence encoding SEQ ID 89. SEQ ID 90 uses the
common abbreviations for nucleotides.

SEQ ID 91

MPLLLLLPLLWAGALAMDKLASGTLEDISRPVCWAAADPGYMERWASGGDPIE
GRGGGGGDPKSCDKPHTCPLCPAPELLGGPSVFLFPPKPKDTLMISRTPEVTCVV
VDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVK
GFYPSDIAVEWESNGQPENNYKATPPVLDSDGSFFLYSKLTVDKSRWQQGNVFS
CSVMHEALHNHYTQKSLSLSPGK

SEQ ID 92

| | |
|---|---|
| 1 | ATGCCGCTGC TGCTACTGCT GCCCCTGCTG TGGGCAGGGG CCCTGGCTAT |
| 51 | GGATAAGCTT GCTAGCGGTA CCCTCGAGGA TATCTCTAGA CCAGTGTGCT |
| 101 | GGGCGGCCGC GGATCCAGGA TACATGGAGC GCTGGGCTAG CGGAGGGGAT |
| 151 | CCCATCGAAG GTCGTGGTGG TGGTGGTGGT GATCCCAAAT CTTGTGACAA |
| 201 | ACCTCACACA TGCCCACTGT GCCCAGCACC TGAACTCCTG GGGGGACCGT |
| 251 | CAGTCTTCCT CTTCCCCCCA AAACCCAAGG ACACCCTCAT GATCTCCCGG |
| 301 | ACCCCTGAGG TCACATGCGT GGTGGTGGAC GTGAGCCACG AAGACCCTGA |
| 351 | GGTCAAGTTC AACTGGTACG TGGACGGCGT GGAGGTGCAT AATGCCAAGA |
| 401 | CAAAGCCGCG GGAGGAGCAG TACAACAGCA CGTACCGTGT GGTCAGCGTC |
| 451 | CTCACCGTCC TGCACCAGGA CTGGCTGAAT GGCAAGGAGT ACAAGTGCAA |
| 501 | GGTCTCCAAC AAAGCCCTCC CAGCCCCCAT CGAGAAAACC ATCTCCAAAG |
| 551 | CCAAAGGGCA GCCCCGAGAA CCACAGGTGT ACACCCTGCC CCCATCCCGG |
| 601 | GATGAGCTGA CCAAGAACCA GGTCAGCCTG ACCTGCCTAG TCAAAGGCTT |
| 651 | CTATCCCAGC GACATCGCCG TGGAGTGGGA GAGCAATGGG CAGCCGGAGA |
| 701 | ACAACTACAA GGCCACGCCT CCCGTGCTGG ACTCCGACGG CTCCTTCTTC |
| 751 | CTCTACAGCA AGCTCACCGT GGACAAGAGC AGGTGGCAGC AGGGGAACGT |
| 801 | CTTCTCATGC TCCGTGATGC ATGAGGCTCT GCACAACCAC TACACGCAGA |
| 851 | AGAGCCTCTC CCTGTCTCCG GGTAAATGA |

SEQ ID 92 is the nucleotide sequence encoding SEQ ID 91. SEQ ID 92 uses the
common abbreviations for nucleotides.

Abbreviations:
Ac—acetyl,
Fam—fluorescein-carbonyl (isomer 5, or isomer 6, or mixture of isomers 5 and 6)
-amide —NH₂ as C-terminal amide group
A alanine
a D-alanine
R arginine
r D-arginine
D aspartic acid
d D-aspartic acid
N asparagine
n D-asparagine
C cysteine
c D-cysteine
E glutamic acid
e D-glutamic acid
Q glutamine
q D-glutamine
G glycine
H histidine
h D-histidine
I isoleucine
i D-isoleucine
L leucine
l D-leucine
K lysine
k D-lysine
M metionine
m D-metionine
F phenylalanine
f D-phenylalanine
P proline
p D-proline
S serine
s D-serine
T threonine
t D-threonine
w tryptophan
w D-tryptophan
Y tyrosine
y D-tyrosine
V valine
v D-valine

### EXAMPLES

### Example 1

S100A4 and Annexin II Co-Expression in Microarray Experiments.

Functionally related genes, i.e. genes participating in common pathways and/or forming multi-protein complexes, are believed to be frequently co-expressed. Co-expression of genes predicts their common transcriptional regulation. Analysis of five publicly available microarray data (Alizadeh et al. 2000 Nature 403: 503-11; Ross et al. 2000 Nature Genetics 24: 227-34; Su et al. 2001 Cancer Res. 15:7388-93; Boldrick et al. 2002 PNAS 99:972-77; Golub et al. 1999 Science 286:531-7; Su et al. 2002 PNAS 99: 4465-70) was performed and genes co-expressed with S100A4 were identified. Each microarray experiment included between 60 and 180 different samples and between 8000 and 18000 analyzed genes. In total, 122 co-expressed genes were found, from which 18 genes, including Annexin II, were identified in two experiments. Gene Ontology (GO) annotations for genes co-expressed with S100A4 demonstrated that 75% of them are membrane-bound or secreted proteins and 43% of the co-expressed genes participate in tumor invasiveness and metastasis. These results strongly suggest that the genes that are co-expressed with S100A4 may participate in common cellular functions such as cancer progression metastasis formation, angiogenesis and some other events.

### Example 2

Cloning of Genes and Expression of Recombinant Proteins.

S100A4 was cloned from human placenta total RNA using RT-PCR approach. Forward primer (CTGGATCCTGTCATGGCGTGCCCTCT) contained BamH I site and corresponds to amino terminal end of protein (SEQ ID 28). Reverse primer (AGGGATCCAACCACATCAGAGGA) contained BamH I site and corresponds to carboxyl terminal end of protein. RT-PCR product was gel purified and ligated into BamH I restricted pGEX-4T-1 vector (Amersham).

Full-sized Annexin II was cloned from human placenta total RNA using RT-PCR approach. Forward primer (GC GGA TCC CCG TCT ACT GTT CAC GAA ATC CTG TG) contained BamH I site and corresponds to amino terminal end of mature protein (without leading methionine) (SEQ ID 29). Reverse primer (T GCG GTC GAC GTC ATC TCC ACC ACA CAG GTA CA) contained Sal I site and corresponds to carboxyl terminal end of protein. RT-PCR product was gel purified and ligated into BamH I/Sal I restricted pGEX-4T-1 vector (Amersham).

N-terminus of Annexin II contains overlapping binding sites for S100A10 (aa 1-14) and tissue type plasminogen activator (t-PA) (aa 7-12). Annexin II is also a major cellular substrate for serine protein kinase C (PKC) and tyrosine protein kinase pp60-Src, amino acids S25 and Y23, respectively. To understand the significance of N-end of Annexin II in its interaction with S100A4, two mutant forms of Annexin II were cloned. In A2del15 (SEQ ID 30) first 15 N-terminal amino acids were deleted, in A2del26 (SEQ ID 31) first 26 N-terminal amino acids were deleted. First mutant does not contain binding sites for S100A10 and t-PA, while second mutant additionally does not contain sites for serine and tyrosine phosphorylation. Truncated forms of Annexin II were cloned by RT-PCR approach using forward primers with BamH I sites, (GC GGA TCC CCT CAC TCT ACA CCC CCA AGT GC) for A2del15 and (GC GGA TCC CCG GTC AAA GCC TAT ACT AAC TTT GAT G) for A2del26, respectively. Reverse primer was the same as for cloning of full-size protein. RT-PCR products were gel purified and ligated into BamH I/Sal I restricted pGEX-4T-1 vector (Amersham).

S100A10 was cloned from human placenta total RNA using RT-PCR approach. Forward primer (GC GGA TCC ATG CCA TCT CAA ATG GAA CAC) contained BamH I site and corresponds to amino terminal end of protein (SEQ ID 32). Reverse primer (CCG CTC GAG TTC TGC CTA CTT CTT TCC CTT C) contained Xho I site and corresponds to carboxyl terminal end of protein. RT-PCR product was gel purified and ligated into BamH I/Xho I restricted pGEX-6P-1 vector (Amersham).

E. coli cells, strain ER2507 (NEB), were made competent by CaCl₂ method and transformed with the corresponding ligation mix. Ten clones were sequenced in both directions and clones without mutations were chosen for protein expression.

For expression of GST-fusion proteins, 400 ml of LB medium were inoculated with 4 ml of overnight culture and cultivated until OD₆₀₀ reached 0.5-0.6. IPTG was then added up to 1 mM, cell culture was cultivated for additional 4 h, after which cells were collected by centrifugation for 15 min at 5000 g and frozen. For lysis, cells were resuspended in 20 ml of PBS buffer containing 1 mM DTT, 1 mM PMSF, and lysozyme and incubated on ice for 30 min. Cell lysate was clarified by centrifugation at 10 000 g for 20 min and 2 ml, bed volume, of glutathione beads (Amersham) were then added and incubated with lysate for 2 h at +4 C with rotation. Beads were collected by centrifugation for 5 min at 600 g and washed 3-4 times with 40 ml of PBS, 1 mM DTT at +4 C on rotator for 20 min. In some experiments glutathione beads with bound GST-fusion proteins were used, in some experiments proteins without tag were used.

To produce proteins without GST tag, 2 ml of PBS and 20 U of thrombin (Amersham) for proteins cloned in pGEX-4T-1 vector or 20 U of PreScission protease (Amersham) for proteins cloned in pGEX-6P-1 vector were added to beads after the last wash and cleavage was performed overnight at room temperature on rotator. Thrombin was removed from protein samples with help of HiTrap Benzamidine FF column, according to manufacturer's recommendations (Amersham). Concentration of protein was measured by Dc Protein Assay (Bio-Rad). If necessary, protein samples were concentrated using Microcon YM-10 centrifugal filter devices (Amicon). Quality of proteins was estimated by SDS-PAAG followed by Coomassie staining (Bio-Rad) or by Western blot with corresponding primary antibody and secondary antibody coupled to horse reddish peroxidase (BD Biosciences Pharmingen). Luminescence was detected with help of Lumi-Light Western blotting substrate (Roche).

### Example 3

Fc-Peptide SEQ ID 5 Recombinant Protein: Construction, Expression, Production and Characterization.

DNA encoding the peptide SEQ ID 5, synthesized by Integrated DNA Technologies, Inc, USA, so that it was flanked by BamH 1 restricted sites enabling ligation into the expression vector. This DNA (pGAT CCA GGA CTG CGC GAG GTG TAC GGA GCG GGC CTT GCC GGG TAC ATG GAG CGC TGG GCT AGC GGA GGG) was cloned into vector Signal plgplus (R&D Systems), which contains an upstream CD33 signal peptide sequence necessary for the secretion of chimera and a human IgG Fc fragment downstream under CMV promoter. The construct was then sequenced with the primer TGCTGGGCGGCCGCGGATCCA and primer TTATGCACCTCCACGCCGTCCACGTA to confirm the open reading frame and the absence of mutations. In order to obtain high level of secretion, DNAs encoding the peptide SEQ ID 5-Fc fusion protein or Fc tail alone were subcloned by PCR using primers CD33-Fw CACCATGCCGCTGCTGCTACTGCTGCCCCT and Fc-Rev AGCTCATTT ACCCGGAGACAGGGAGAGGCTCTTCT, into the expression vector pcDNA/V5/GW/D-TOPO (Invitrogen) according to the manufacture's instruction. Sequencing confirmed that no errors had been introduced by PCR.

For protein expression, Chinese hamster ovary (CHO) cells were transfected with Fc-fusion protein construct or Fc protein without peptide insert by the use of Lipofectamin 2000 (Invitrogen) and grown for 24 hr in Ham's F-12 medium (Invitrogen) containing 10% fetal calf serum. The following day, cell cultures were treated with the selection antibiotic Geneticin (Invitrogen) at 1 mg/ml medium. Stable clones were selected by limiting dilution and screened for Fc expression.

For large-scale production, conditioned media were collected from cultures during 10 days in presence of 10 mM sodium butyrate to stimulate protein expression. For purification, the Fc-fusion protein bound on Protein A-Sepharose (Amersham Biosciences) was eluted by 0.1 M glycine-HCl (pH 2.5) and immediately neutralized with 1 M Tris (pH 9.5). Purified protein was concentrated using centrifugal filter devices (Millipore, Bedford, Mass.), dialyzed against PBS, and kept frozen in aliquots. The protein concentration of purified Fc-fusions was determined by the BCA assay. The chimeric protein was further characterized by SDS-PAAG followed by Coomassie staining and Western blotting. The product of 303 amino acids was visualized on SDS-PAAG as one band of 35 kDa and contained: CD33 signal sequence (position 1-18), peptide MT7 (position 41-56) and hlgG1 Fc tail (position 72-303). The product fusion protein has amino acid sequence SEQ ID 39, and is encoded by DNA sequence SEQ ID 88.

The fusion protein SEQ ID 89, which is encoded by DNA sequence SEQ ID 90, and the fusion protein SEQ ID 91 which is encoded by DNA sequence SEQ ID 92, can also be prepared using the methods described in this Example, while the respective procedures start with the synthesis of DNA encoding the peptide SEQ ID 14, and peptide SEQ ID 12, respectively.

### Example 5

Immune-Co-Precipitation of S100A4 and Annexin 2, its Truncated Fragments and Analysis of the Peptide Effects on It

I. Formation S100A4 Aggregates and Their Dissociation.

To force the aggregate formation, S100A4 at the concentration of 0.5 mg/ml in TBS containing either 5 mM Ca2+, or 5 mM EGTA, was incubated overnight at 37° C. The resulted mixture was analyzed by 15% SDS/PAAG electrophoresis in the absence of reducing agents.

To dissociate the S100A4 aggregates, they were treated at 37° C. overnight with 5 mM DTT; 5 mM EGTA or their combination. The resulted mixtures were analyzed by 15% SDS PAAG.

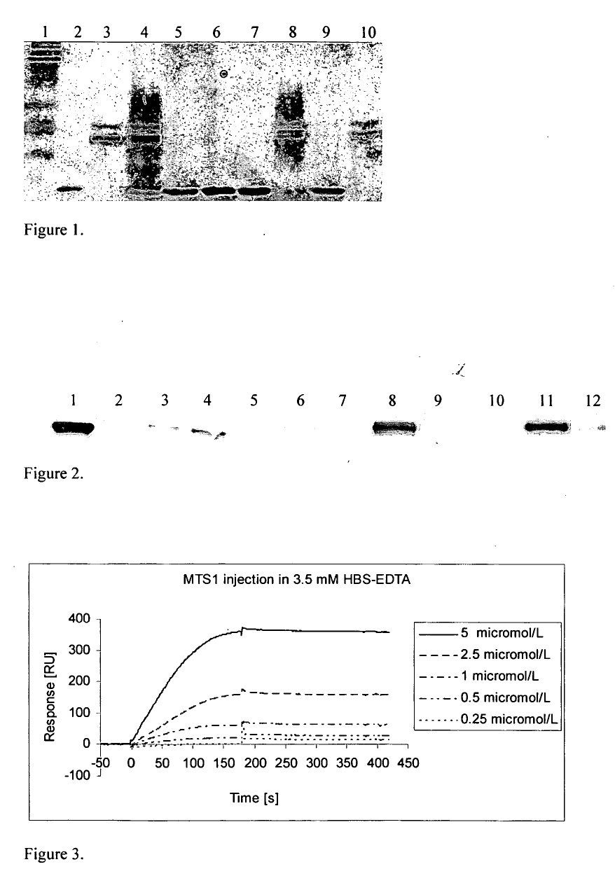

The results shown in FIG. 1 revealed that in the presence, and in the absence of Ca2+, S100A4 forms aggregates of multiple compositions with dimeric form of the protein dominating in both cases. The aggregates remained stable and did not chang their elictrophoretic mobility at non-reducing conditions, both in the presence of Ca2+ or EGTA. However, in the presence of 5 mM DTT a complete dissociation of the aggregates was observed.

II. Analysis of the S100A4-Annexin II Complex Formation by Immune-Precipitation

S100A4 and annexin II proteins, both at the concentrations of 1.5 mg/ml, were pre-incubated overnight at 37° C. in TBS containing either 5 mM CaCl₂, or 5 mM EGTA.

The formation of the S100A4-annexin II complex was analyzed by immunoprecipitation with an anti-S100A4 rabbit polyclonal antibody. To this end, the reaction mixture was incubated with the antibody (10 μg/ml) for 1 h at 37° C. and then precipitated by adding protein G-sepharose beads (Affi-Gel; Bio-Rad Laboratories, Hercules, Calif.) and incubating the resulted mixture for 1 h at room temperature. After that the beads were washed 3 times by 500 ul of TBS, boiled in Laemmli sample buffer for 5 min, and then loaded on SDS-PAAG. The proteins were then electro-transferred to a nitrocellulose membrane. The Western blot analysis was performed using anti-Annexin II antibody (BD Pharmingen) used at 1:5000 dilution and sheep anti-mouse antibody conjugated to horseradish peroxidase (Amersham Pharmacia Biotech, Piscataway, N.J.) used at 1:10000 dilution, followed visualization of the Annexin II bands by chemiluminescence.

The results shown on FIG. 2 indicate that S100A4 both in the monomer and aggregated forms (prepared either in the presence of Ca2+, or in the presence of EGTA) is capable of complex formation with annexin II. However, the complex formation is more efficient in the case when the aggregated S100A4 are used. Additionally, the interaction between S100A4 (both monomer and oligomer) and annexin II is more efficient in the absence of Ca2+ (EGTA conditions) compared to that observed in the presence of Ca2+.

### Example 6

Analysis of Binding of S100A4 and Annexin 2 by Surface Plasmon Resonance (BIAcore)

A. Immobilization of Annexin 2.

Annexin 2 has been coupled to the surface of CM5 sensor chip in BIAcore 1000 using standard manufacturers procedure. The surface of a cell has been activated with a 0.2 M N-ethyl-N-(3-diethylaminopropyl) carbodiimide, 0.05 M N-hydroxysuccinimide solution, followed by injection of 25 microgram/mL solution of Annexin 2 in acetate buffer pH 4.5. 412 RU of Annexin 2 have been immobilized. The unreacted activated groups were blocked with 1 M ethanolamine hydrochloride. The surface of the control cell was activated and blocked using the same procedure.

B. Analysis of Interaction of Annexin 2 with S100A4 in Absence of Calcium

The HBS-EP flow buffer consisting of 10 mM HEPES (pH 7.4), 150 mM NaCl, 3.4 mM EDTA, 0.005% P20, was filtered through 0.2 micrometer filter and degassed. A series of solutions of S100A4 in HBS-EP of the concentrations 0.25, 0.5, 1, 2.5, and 5 micromol/L, have been injected into cell with immobilized Annexin 2, and into the control cell, at the flow rate 30 microliter/min, contact time 180 s, followed by HBS-EP for at least 240 s. Sensograms (response units [RU] versus time) have been recorded, and for each S100A4 sample the response recorded in control cell have been subtracted from that in cell with immobilized Annexin 2. The results are presented in the FIG. 3. The surface of the cell was regenerated after each injection of S100A4 using HBS buffer containing 25 mM CaCl₂, followed by re-equilibration with HBS-EP.

C. Analysis of Interaction of Annexin 2 with S100A4 in the Presence of Calcium

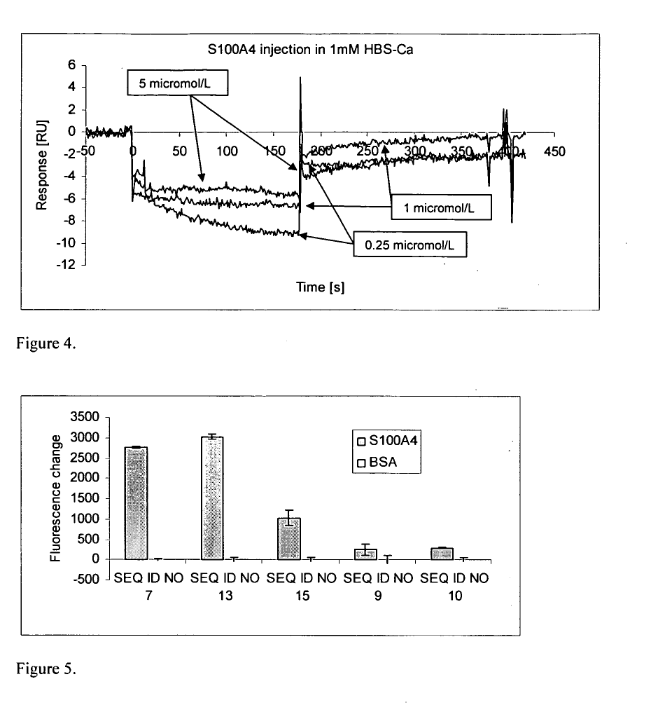

The HBS-Ca flow buffer consisting of 10 mM HEPES (pH 7.4), 150 mM NaCl, 1 mM CaCl₂, 0.005% P20, was filtered through 0.2 micrometer filter and degassed. A series of solutions of S100A4 in HBS-Ca of the concentrations 0.25, 1, and 5 micromol/L, have been injected into cell with immobilized Annexin 2, and into the control cell, at the flow rate 30 microliter/min, contact time 180 s, followed by HBS-Ca for at least 240 s. Sensograms (response units [RU] versus time) have been recorded, and for each S100A4 sample the response recorded in control cell have been subtracted from that in cell with immobilized Annexin 2. The results are presented in the FIG. 4. The surface of the cell did not require regeneration.

### Example 7

Solid Phase Peptide Synthesis of the Peptides SEQ ID NO 6, 7, 8, 9, 10, 11, 13, 15, 38.

For each synthesis the starting material was 0.32 g (0.2 mmol) of Rink Amide resin (4-(2′,4′-Dimethoxyphenyl-Fmoc-aminomethyl)-phenoxy resin) substituted at a level of 0.63 mEq per gram of resin (Nova Biochem, CA). Each of the amino acids, starting with the respective C-terminal, was added in sequence in a synthesis cycle consisting of the three steps: of: piperidine deprotection (step 1), coupling (step 2), and ninhydrine test (step 3). If the test showed incomplete coupling, the coupling step was repeated. The synthesis of peptides SEQ ID NO 6, and 38 was completed with Fmoc-deprotection (step 1), and acetylation (step 4). The synthesis of peptides SEQ ID NO 7, 8, 9, 10, 13, and 15 was completed with Fmoc-deprotection (step 1), and labeling with fluoresceine (step 5). The synthesis of peptides SEQ ID NO 11 was completed with only Fmoc-deprotection (step 1). All the completed peptides were subjected to trifluoroacetic acid cleavage (step 6) and then purified (step 7). All the operations were performed in a 20 mL polypropylene reactor with a polyethylene frit for draining the solvent. The resin was agitated with the solvents and the respective solutions using a shaker rotating the reactor for 180 degree.

1. Fmoc-Deprotection

The Fmoc-protecting group was removed from the starting resin, or from the α-amino nitrogen of the amino acid previously attached to the resin, by treating the resin twice with 20% piperidine in dimethylformamide (DMF) (10 mL) for 3 min, and a second time for 17 min. The resin was then washed six times with 5 mL of DMF, each wash taking one minute.

2. Coupling

The appropriate Fmoc-protected amino acid (1.2 mmol dissolved in 3.5 mL DMF), benzotriazole-1-yl-oxy-tris-pyrrol idino-phosphonium hexafluorophosphate (PyBOP) (0.625 g dissolved in 1.5 mL DMF), and diisopropylethylamine (0.42 mL) was added to the resin, and the mixture was agitated for 90 minutes. The resin was washed four times with 5 mL DMF.

The amino acid derivatives used are represented in

TABLE 2

| Amino acid | Derivative |
|---|---|
| Ala | Fmoc-Ala-OH |
| D-Ala | Fmoc-D-Ala-OH |
| Arg | Fmoc-Arg(Pbf)-OH |
| D-Arg | Fmoc-D-Arg(Pbf)-OH |
| Asp | Fmoc-Asp(OtBu)-OH |
| D-Asp | Fmoc-D-Asp(OtBu)-OH |
| Cys | Fmoc-Cys(Trt)-OH |
| Gln | Fmoc-Gln(Trt)-OH |
| Glu | Fmoc-Glu(OtBu)-OH |
| D-Glu | Fmoc-D-Glu(OtBu)-OH |
| Gly | Fmoc-Gly-OH |
| His | Fmoc-His(Trt)-OH |
| D-His | Fmoc-D-His(Trt)-OH |
| Ile | Fmoc-Ile-OH |
| D-Ile | Fmoc-D-Ile-OH |
| Leu | Fmoc-Leu-OH |
| D-Leu | Fmoc-D-Leu-OH |
| Lys | Fmoc-Lys(Boc)-OH |
| D-Lys | Fmoc-D-Lys(Boc)-OH |
| Met | Fmoc-Met-OH |
| D-Met | Fmoc-D-Met-OH |
| Phe | Fmoc-Phe-OH |
| D-Phe | Fmoc-D-Phe-OH |
| Pro | Fmoc-Pro-OH |
| Ser | Fmoc-Ser(tBu)-OH |
| D-Ser | Fmoc-D-Ser(tBu)-OH |
| Tyr | Fmoc-Tyr(tBu)-OH |
| D-Tyr | Fmoc-D-Tyr(tBu)-OH |
| Trp | Fmoc-Trp(Boc)-OH |
| D-Trp | Fmoc-D-Trp(Boc)-OH |
| Thr | Fmoc-Thr(tBu)-OH |
| Val | Fmoc-Val-OH |
| D-Val | Fmoc-D-Val-OH |

3. Ninhydrine Test

A small sample of the resin (approxymatly 30 beads) was transferred to a test tube. One drop of 1% ninhydrin solution in ethanol, one drop of 80% aqueous phenol, and one drop of 0.001% KCN in pyridine were added to the sample of resin, and the mixture was heated to 120° C. for 5 min. Blue color of beads showed incomplete coupling. In this case the coupling step 2 was repeated. If complete, the synthesis proceeded to the next cycle.

4. Acetylation

After completion of the last cycle with N-terminal amino acid, and Fmoc-deprotection, the resin was agitated with acetic anhydride (0.11 mL) and diisopropylethylamine (0.42 mL) in 2.5 mL DMF for 90 minutes at room temperature.

5. Labeling with Fluorescein

After completion of the last cycle with N-terminal amino acid, and Fmoc-deprotection, the resin was agitated with 5(and 6-)-carboxyfluorescein (0.45 g dissolved in 3 mL DMF), PyBOP (0.625 g dissolved in 2 mL DMF), and diisopropylethylamine (0.42 mL) for 120 min.

6. Cleavage with Trifluoroacetic Acid

The resin was washed 6 times with DMF, twice with DMF/methanol (1:1 v/v), and three times with methanol, and dried in vacuum for 1 hour. A mixture of trifluoroacetic acid (TFA, 4.5 mL), water (0.25 mL), ethanedithiol (0.125 mL) and triisopropylsilane (0.125 mL) was added to the dry resin, and was agitated for 2 hours. The liquid was drained, and the resin was washed with 2 mL TFA. Combined liquids were evaporated in a stream of dry nitrogen. The residue was washed twice with 10 mL of anhydrous ether, and the crude products were dissolved with a 1:1 mixture of acetonitrile and water (10 mL), and freeze dried.

7. Purification

The lyophilized powder was dissolved in a mixture CH₃CN:H₂O (1:1, v/v, 10 mg of crude peptide in 2 mL) and loaded onto a Vydac C18, preparative column (25×2.25 cm). The loaded column was eluted with a gradient of two-component eluent, starting from 10% of solution B in solution A, at flow rate 5 mL/min, and changing the solvent composition 1% per minute. Solution A was 0.1% TFA in H₂O, and solution B was 0.1% TFA in CH₃CN. Fractions were identified by electro-spray mass spectrometry, as defined in the Table 3 below. Fractions exhibiting purity equal to or better than that desired were pooled and lyophilized. The product was dissolved in CH₃COOH:H₂O (1:1, v/v, 1 mL per 1 mg of product) to render the purified final product as the acetate salt.

TABLE 3

| SEQ ID NO | m/e |
|---|---|
| 6 | 2101 |
| 7 | 1209 (double charge) |
| 8 | 1261 (double charge) |
| 9 | 1209 (double charge) |
| 10 | 1224 (double charge) |
| 11 | 2058 |
| 13 | 1300 |
| 15 | 1166 |
| 38 | 2130 |

The methods of synthesis described in this example is also useful for the synthesis of peptides SEQ ID 5, 12, 14, 33-37, and 41-87; these syntheses require that Fmoc protected Wang resin preloaded with the C-terminal amino acid is used instead of the Rink amide resin.

### Example 8

Analysis of Interaction of Peptide SEQ ID NO 7, 9, 10, 13 and 15 with S100A4 by Fluorescence Assay

10 mM solution of each of the tested peptide in DMSO was diluted with TBS-Ca buffer (TRIS buffer containing 150 mM NaCl, 0.05% bovine serum albumine BSA, 2.5 mM CaCl₂), to obtain final peptide concentration 5 micromol/L. Samples of these peptide solutions were placed in a 96 well plate (Falcon No. 3353910), 90 microliters per well. Fluorescence of these samples was determined using FL600 Fluorescence plate reader (BioTek) equipped with excitation filter 485 nm and emission filter 530 nm. 10 microliters of 30 micromol/L solution of S100A4 in TBS-Ca was added to each of the peptide samples. Each sample was prepared in triplicate. The samples were mixed carefully while adding the protein to the peptide, and then they were incubated for 10 h at room temperature. The fluorescence of these samples, as well as the control samples containing 0.05% BSA, was determined using the same conditions as for the peptide samples before adding S100A4, and the change of the fluorescence intensity was calculated individually for each sample. The results, average of triplicates, are presented in the FIG. 5.

### Example 9

Analysis of Binding of Peptide SEQ ID NO 7, 9, and 10 with S100A4 by Fluorescence Polarization Assay

The following working buffers have been prepared:

TBS-EGTA: 10 mM TRIS buffer containing 150 mM NaCl, 0.05% bovine serum albumine (BSA), 1 mM EGTA

TBS-Ca: 10 mM TRIS buffer containing 150 mM NaCl, 0.05% bovine serum albumine (BSA), 1 mM CaCl₂

10 mM solution of peptide SEQ ID NO 7 in DMSO was diluted with in either TBS-EGTA or TBS-Ca to obtain final peptide concentration 20 nM. Samples of these peptide solutions were placed in 384 well plate (Costar No. 3710), 45 microliters per well. Fluorescence polarization of such samples was determined using Perkin Elmer Wallac EnVision 2100 Multilabel Reader equipped with excitation filter 485 nm and emission filters 535 nm for each channel.

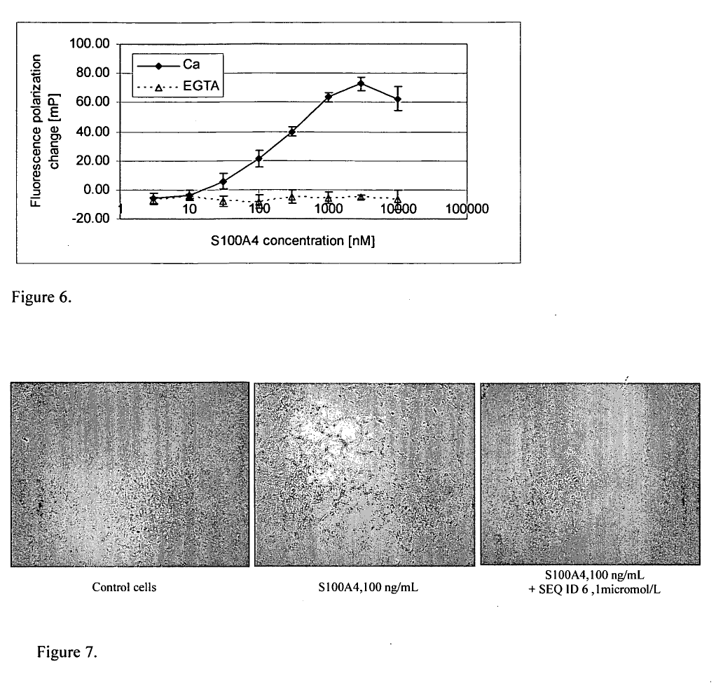

S100A4 has been diluted by either TBS-EGTA or by TBS-Ca to obtain a series of solutions of such concentrations that after adding 5 microliters each solution to 45 microliter peptide samples the final concentration of S100A4 was one of following: 0, 10, 30, 100, 300, 1000, 3000, or 10000 nM. Each sample was prepared in triplicate. The samples were mixed carefully while adding the protein to the peptide, and then they were incubated for 1 h at room temperature. The fluorescence polarization of the samples was determined using the same conditions as for the peptide samples before adding S100A4, and the change of fluorescence polarization of the peptide SEQ ID NO 7 upon adding S100A4 was calculated individually for each sample. The results, average of triplicates, are presentsd on the FIG. 6. The apparent binding constant of peptide SEQ ID NO 7 binding to S100A4 is 236 nM.

The same procedure and conditions have been applied to test binding of peptide SEQ ID NO 9, and peptide SEQ ID NO 10, to S100A4. No change of fluorescence polarization has been detected for these peptides.

### Example 10

Inhibition of S100A4 Induced Angiogenesis Peptide SEQ ID 6

Human cerebral endothelial cells (HCEC) were isolated using previously described protocols (Stanimirovic, D., P. Morley, et al. (1996). “Angiotensin II-induced fluid phase endocytosis in human cerebromicrovascular endothelial cells is regulated by the inositol-phosphate signaling pathway.” J Cell Physiol 169(3): 455-67.). Purity of HCEC cultures generated by these procedures was routinely assessed by the immunocytochemical staining for Factor VIII-related antigen and the lack of staining for smooth muscle β-actin, and is estimated to be >95%. The morphological, phenotypic, biochemical and functional characteristics of these HCEC cultures have been described in detail previously (Muruganandam, A., L. M Herx, et al. (1997). “Development of immortalized human cerebromicrovascular endothelial cell line as an in vitro model of the human blood-brain barrier.” Faseb J 11(13): 1187-97).

Capillary Tube Formation in Matrigel™

HCEC were seeded (8×10⁴ cells) on a semi-solid basement membrane matrix, Matrigel™ (Collaborative Biomedical Products, Bedford, Mass.)-coated 24-well plates and exposed to serum-free media containing various treatments. Cells were treated with 10 nM VEGF₁₆₅ or glioblastoma cell (U-87MG, ATCC, Rockville, Md.)-conditioned media (serum-free DME conditioned for 48-72 h) in the absence or presence of SP5.2 or control peptide for 24 hours. HCEC were then labelled with a vital dye, calcein-AM (2 μM) for 30 minutes. Cells were then washed in PBS and capillary-like tube formation, a measure of in vitro angiogenesis, was observed using a Zeiss LSM 410 inverted laser scanning microscope (Thornwood, N.Y.) with an Argon/Krypton ion laser and a Zeiss LD achroplan 5×, 0.6 NA objective. Confocal apertures for each recorded wavelength were adjusted to a full width half maximum of 20 μm. The emitted light was collected through a 515-540 nm band-pass filter using an excitation of 488 nm. Eight to 32 individual optical sections were collected and the average was calculated. Standard image processing was performed to enhance brightness and contrast using ImageSpace software (Molecular Dynamics Inc., Sunnyvale, Calif.).

S100A4 protein induces angiogenesis in a primary HCEC grown in Matrigel™, at the concentrations of S100A4 starting from 10 nM. The peptide SEQ ID NO 6 was found to completely inhibit S100A4-induced formation of capillary-like tubes (FIG. 7).

### Example 11

S100A4 Accelerates t-PA-Dependent Conversion of Plasminogen to Plasmin in Ca²⁺-Independent Manner.

Non-active plasminogen can be converted to active protease plasmin by tissue type (t-PA) or urokinase type (u-PA) plasminogen activators. Except for its well-known fibrinolytic activity, plasmin also directly and indirectly, by activation of matrix metalloproteinases (MMPs), participates in the degradation of extracellular matrix that is important for the angiogenesis and tumor invasion. The kinetics of t-PA-mediated plasminogen activation was determined by measuring amidolytic activity of the plasmin generated during plasminogen activation. The reaction was performed with the plasmin substrate Chromozyme PL, tosyl-glycyl-prolyl-lysin-4-nitranilide-acetate (Roche) in the final concentration of 125 μM in a buffer consisting of 50 mM Tris-HCl, pH 7.4, 100 mM NaCl, 5 mM of CaCl₂ or 5 mM of EGTA, and 150 nM of [Glu] plasminogen (Calbiochem). The reaction was preincubated with or without 2.5 μM S100A4 at 24 C° for 1 h in 96-well plates. The reaction was initiated by addition of t-PA (Calbiochem) up to 5 nM concentration, final volume 100 μl. Kinetic of substrate hydrolysis was measured at 2 min intervals at 405 nm in Spectramax Plus 384 spectrophotometer (Molecular Devices). Analysis of kinetics curves (Vmax, time to Vmax, and initial slope of kinetic data converted to A₄₀₅ versus time²) was performed with the help of Softmax PRO program (Molecular Devices).

Kinetics of plasminogen to plasmin conversion consists of three parts. As shown in FIG. #3 curves 1 and 2 for t-PA, during first 1500 s the reaction is second order that corresponds to conversion of plasminogen to plasmin and consequent cleavage of chromogenic substrate by plasmin. The second part of reaction is linear, for which Vmax can be calculated. It starts after total conversion of plasminogen to plasmin and corresponds to cleavage of chromogenic substrate by plasmin with constant rate. In the third part of reaction, after 2700 s, the reaction is slowing down that corresponds to depletion of chromogenic substrate and finally the reaction reaches a plateau when all substrate is cleaved. As follows from the comparison of curves 1 and 2, t-PA along converted plasminogen to plasmin equally efficiently in the presence or absence of calcium. Neither conversion of plasminogen to plasmin took place in the absence of t-PA no S110A4 induced such conversion by itself, curves 5 and 6. In the presence of S100A4 linear part of reaction starts substantially earlier, which means that total conversion of plasminogen to plasmin was accelerated. Kinetic parameters for t-PA along and for combination of t-PA with S100A4 are represented in Table 4. Data received demonstrated that S100A4 does not substantially change the Vmax of reaction, but rather change the time at which the reaction reaches the maximum rate. Time to Vmax decreased from 2200 sec to 1440 sec and initial rate of plasmin generation increased more than 3-fold from 0.078 to 0.250. S100A4 accelerated conversion of plasminogen to plasmin in calcium independent manner, curves 3 and 4.

TABLE 4
Kinetic parameters of t-PA-dependent conversion of plasminogen to plasmin.
Effect of S100A4.

| Kinetic | t-PA | | t-PA + S100A4 | |
|---|---|---|---|---|
| parameter | Ca | EGTA | Ca | EGTA |
| Vmax, ΔA₄₀₅/t(min) * 1000 ± SD | 7.152 ± 0.130 | 8.589 ± 0.342 | 8.420 ± 0.227 | 8.552 ± 0.328 |
| Time to Vmax, t(sec) ± SD | 2200 ± 69 | 2102 ± 69 | 1440 ± 10 | 1440 ± 120 |
| Slope, | 0.078 ± 0.007 | 0.080 ± 0.008 | 0.250 ± 0.016 | 0.238 ± 0.027 |
| ΔA₄₀₅/t²(min²) * 1000 ± SD | | | | |

### Example 12

Modulation of S100A4 Dependent tPA-Mediated Conversion of Plasminogen to Plasmin by Peptides SEQ ID 6 and SEQ ID 38 and Chimera SEQ ID 39.

The effects of peptide SEQ ID 6, peptide SEQ ID 38 and chimera SEQ ID 39 on acceleration oft-PA-mediated conversion of plasminogen to plasmin induced by S100A4 were measured. The kinetics of t-PA-mediated plasminogen activation were determined by measuring the amidolytic activity of plasmin formed during activation of plasminogen. All the experiments were done in 100 μl volume in 96-well plates in triplicates. Glu-plasminogen (Calbiochem) at the concentration of 100 nM was preincubated at 25° C. for 1 h in the presence or absence of 1 μM recombinant S100A4 in a buffer consisting of 50 mM Tris-HCl, pH 7.4, 100 mM NaCl and 5 mM CaCl₂ or 5 mM EGTA. In some experiments, different concentrations of peptides or chimera were preincubated together with proteins. The reaction was initiated by addition of 5 nM t-PA (Calbiochem) and 125 μM chromogenic plasmin substrate, Chromozym PL (Roche). The reactions were monitored at 405 nm for 1.5 h in a SPECTRAmax PLUS³⁸⁴ microplate spectrophotometer (Molecular Devices) under control of the SOFTmax PRO kinetic program (Molecular Devices). The initial rates of plasmin generation, during the first 10-15 min, were calculated using linear regression analysis of plots of A_{405 nm} versus time 2. The initial rates of plasmin generation were reported in units (K) of A_{405 nm}/min²×10³. In the absence of t-PA, plasmin generation did not take place regardless of the presence of S100A4. The titration data were analyzed with the four-parameter logistic equation K=(A−D)/(1+(x/C)^{B})D. If A, asymptotic minimum, =0, this equation is converted to the Michaelis-Menten equation K=D/(1+(C/[S])^{B}), where [S] is substrate concentration, D=Vₘₐₓ, C=Kₘ, and B is the Hill coefficient.

The addition of S100A4 to basal reaction (t-PA plus plasminogen) produced a significant increase in the plasminogen conversion rate. The results indicate that S100A4 accelerated plasmin formation (K units) 15-fold in the presence of Ca²⁺ and 11-fold in the presence of EGTA. The stimulation of the plasminogen conversion by S100A4 was concentration-dependent with the half-maximal effect at 2.4 μM.

The kinetic parameters of t-PA-mediated plasminogen conversion were evaluated in the presence and absence of S100A4 as a function of plasminogen concentrations ranging from 3 nM to 3 μM. The titration curves demonstrated that S100A4 significantly reduced Km of the t-PA-mediated plasminogen conversion from 429 to 59 nM, but did not substantially affect the Vₘₐₓ of the reaction, conferring a 7.6-fold increase in the catalytic efficiency (Vₘₐₓ/Kₘ) of t-PA in the presence of EGTA (Table 5) and 10-fold in the presence of calcium.

TABLE 5
Effect of S100A4 on kinetic parameters of t-PA-dependent
conversion of plasminogen to plasmin.

| Kinetic | t-PA | |
|---|---|---|
| parameter | (basal reaction) | t-PA + S100A4 |
| Vmax | 1.18 | 1.24 |
| Km, μM | 0.429 | 0.059 |
| Vmax/Km | 2.8 | 21.0 |
| Increase in t-PA activity | 1 | 7.6 |

Peptides SEQ ID 6 and SEQ ID 38 inhibited the accelerating effect of S100A4 on t-PA-dependent conversion of plasminogen to plasmin in concentration—dependent manner. Titration of peptides in range 0.1-100 μM demonstrated that IC50 was 6 and 3.5 μM for SEQ ID 6 and SEQ ID 38, respectively, and 15 μM of any of the two peptides totally neglected the effect of S100A4.

On the other hand, chimera SEQ ID 39 additionally accelerated plasmin formation already accelerated by S100A4. Titration of the chimera in range 1-30 μM demonstrated 2-fold increase in S100A4 activity with Km close to 0.5 μM. Control construct, Fc protein without fusion, did not show any substantial effect on S100A4 activity.

### Example 13

Genetic Construct Containing Peptide SEQ ID NO 5 and β-gal

A prokaryotic expression vector, pQE-16 was used to construct the fusion protein MT05-β-gal, containing peptide SEQ ID NO 5 on N-terminus and (His)₆ on C-terminus of β-galactosidase. To this end, the vector pCMV was digested with Bam H1, the resulting β-galactosidase gene was purified from agarose gel and modified by PCR as so to introduce Bgl II at the 3′end for cloning into pQE-16 instead of DHFR, that has been removed by digestion of plasmid with BamH1 and Bgl II. SEQ ID NO 5 insert containing EcoRI site at 5′ end, BamH I site at 3′ end and (Gly)₄ linker was prepared by annealing of two single stranded oligonucleotides, encoded peptide, and inserted in the pQE16-β-gal vector. pQE16-MT05-β-gal was used to transform competent E. coli M15 cells. The resulting clones were sequenced to verify the fusion gene.

The expression of β-galactosidase and the presence of His-tag were verified by Western-blot using anti-β-galactosidase and anti-His-HRP antibody. Fusion protein was purified using Ni-NTA resin.

### Example 14

Conjugation of Peptides SEQ ID NO 8 with Horseradish Peroxidase

The carrier protein, for example horseradish peroxidase (ICN, 250 u/mg) was dissolved in phosphate buffer (0.1M Na₂HPO₄, 0.1 M NaCl, 1 mM EDTA and pH 8.5) at final concentration 3 mg/ml. N-succinimidyl-3-(2-pyridylthio)propionate (SPDP, Sigma Chemical) was dissolved in 0.133 mL of dimethylformamide (DMF), in a proportion of 0.234 mg SPDP=0.039 mL DMF. The solution of SPDP was added to solution of peroxidase and incubated with stirring at room temperature for 30 minutes. After modification, activated protein was purified by gel filtration. The solution of peroxidase was applied to the Sephadex G-25 column (Fisher, 20 mL) and eluted with 50 ml of phosphate buffer. Detect at 280 nm with a sensitivity of 50 and lamp intensity of 0,005 Au. The fractions (1 mL) were collected using a fraction collector (Pharmacia Biotech). The fractions containing modified Peroxidase were selected and combined (total volume of 5-7 mL). Aliquot of 1 mL was kept for the control. Number of activated groups was evaluated by treatment of aliquot of activated protein with 1 mg/mL of L-cysteine methyl ester hydrochloride (Aldrich Chemical). Amount of recovered 2-pyridyl disulphide was measured by UV absorbency at 343 nm. Control sample was treated with cysteine for 15 hours at room temperature, purified by gel filtration and used as a reference in receptor binding assays. The peptide SEQ ID NO 8, 1 mg was dissolved in 200 μl of phosphate buffer. Activated peroxidase was mixed with the peptide and incubated with stirring for 24 hours, at room temperature. The reaction was controlled by UV detection at 343 nm (detection of 2-pyridyl disulphide). The conjugate was purified by gel filtration using Sephadex G-25 column. The conjugate fractions were collected and combined. The protein concentration was determined using Bradford method (Coomasie blue, Bio-Rad) and conjugation was confirmed by SDS/PAAG electrophoresis. Peroxidase activity of the conjugate per mg of protein was determined by incubation of conjugate aliquots with ABTS solution (0.22 mg/ml 2′2′-azino-bis-93′-ethylbenzthiazoline-6-sulphonic acid) diammonium salt, 0.05M citric acid, pH 4.0, 0.05% H₂O₂) for 30 min. at room temperature and detection the absorbance at A405.

### Example 15

Conjugation of Peptides SEQ ID NO 8 with Poloxamer 407

A. Preparation of 4-Nitrophenylchloroformate derivative of Poloxamer 407

53.43 g of dry Poloxamer 407 was added to a 500 ml round bottom flask equipped with a stirrer and gas inlet attached to a cylinder containing dry high purity nitrogen. The flask was flushed with dry nitrogen for 15 minutes. This nitrogen flush was maintained through the reaction. 200 mL of anhydrous methylene chloride was added to the flask, followed by 0.5 g of triethylene amine. After 15 minutes of stirring, a solution of 1.0686 g of 4-nitro phenyl chloroformate in 50 mL of anhydrous methylene chloride was added to the reaction mixture. The reaction mixture was stirred for 18 hours at room temperature, and then it was added drop wise to 2 liters of petroleum ether. The upper layer was decanted and the glue like product was dissolved in 100 mL of anhydrous methylene chloride. This mixture was added drop wise to 2 liters of petroleum ether. Again the upper layer was decanted and the glue like product was washed several times with petroleum ether till fine white powder was obtained. This powder was dried under vacuum and kept there until further used.

B. Preparation of Amino Derivative of Poloxamer 407

30.0 g of 4-nitrophenylchloroformate derivative of Poloxamer 407, prepared by the procedure described above, was dissolved in 115 mL of anhydrous methylene chloride in 250 mL round bottom flask. 0.44 g of ethylene diamine was added to this solution. After stirring the mixture for 20 hours at room temperature, the reaction mixture was added drop wise to 2 liters of petroleum ether. The upper layer was decanted and the glue like product was dissolved in 60 ml of methylene chloride anhydrous. This mixture was added drop wise to 2 liters of petroleum ether. Again the upper layer was decanted and the glue like product was washed several times with petroleum ether till fine yellow powder was obtained. This powder was dried under vacuum. The dry product was dissolved in 400 mL of milli-Q water and was dialyzed against 15 liters of de-ionized water at 4° C. using spectra/Por 3 MWCO 3500 dialysis bags. The milli Q water was changed 12 times every 12 h. After dialysis the product was filtered and freeze-dried, and stored in vacuum until further use.

C. Preparation of Maleimido Derivative of Poloxamer 407

310 mg of amino derivative of Poloxamer 407, prepared by the procedure described above, was dissolved in a mixture of 2 mL of dry dimethylformamide and 0.015 mL of diisopropylethyleamine. The reaction mixture was stirred for 10 minutes. To this reaction mixture was added 25 mg of N-hydroxysuccinimide ester of gama-maleimidobutyric acid. After stirring for 6 hours the reaction mixture was diluted by 20 mL of milli Q water, and was dialyzed against 15 liters of de-ionized water at 4° C. using spectra/Por 3 MWCO 3500 dialysis bags. The milli Q water.was changed 6 times every 12 h. After dialysis the product was filtered and freeze-dried and stored in vacuum until further use.

D. Preparation of Conjugate of Poloxamer 407 and Peptide SEQ ID NO 8

36 mg of maleimide derivative of Poloxamer 407, prepared by the procedure described above, and 2 mg of peptide SEQ ID NO 8, was dissolved in 1 mL of dry dimethylformamide, and was stirred for 4 hours. 3.0 mg of L-cysteine methyl ester hydrochloride was added to the reaction mixture, and it was stirred for 1 more hour. The reaction mixture was diluted by 20 mL of milli-Q water, and was dialyzed against 15 liters of deionized water at 4° C. using spectra/Por 3 MWCO 3500 dialysis bags. The milli Q water was changed 8 times every 12 h. After dialysis the product was filtered, freeze dried and stored in vacuum until further use.

### Example 16

Conjugation of Peptides SEQ ID NO 8 with PEG-1500

The conjugate was prepared using di-amino derivative of polyoxyethylene MW 1500 (PEG-bis-amine MW 1500 from Shearwater Polymers, Inc. Al., Cat. No. PT-017-08). PEG-bis-amine was allowed to react with SPDP (N-Succinimidyl 3-[2-pyridyldithio] propionate, from Sigma, Cat. No. P-3415). The resulting PEG-bis-SS-pyridyl was purified with HPLC. The product was mixed and reacted with excess of peptide. The progress of the conjugation reaction was monitored by light UV absorption at 340 nm. Upon completion, the components of the reaction mixture were separated using HPLC.

A. Preparation of PEG-bis-SS-pyridyl.

PEG-bis-amine (15 mg dissolved in 0.2 mL methanol) was mixed with SPDP (5 mg dissolved in 0.1 mL methanol) and diisopropylethylamine (0.027 mL of 10% solution in methanol). The mixture was stirred for 30 minutes, and then it was loaded onto a Vydac C18 preparative column (25×2.25 cm). The column was eluted with a two-component eluent gradient 0.5% per minute, starting from 0% of solution B in solution A, at flow rate 5 mL/min. Solution A was 0.1% TFA in H₂O, and solution B was 0.1% TFA in CH₃CN. Fractions were identified by electrospray MS, and by increased UV absorption at 340 nm after mixing sample with 1% ethanedithiol in methanol (1:1 v/v). Fractions 50-60 mL after the void volume were pooled together and freeze-dried, and yield 6.3 mg of PEG-bis-SS-pyridyl.

B. Conjugation of PEG-bis-SS-pyridyl with Peptide SEQ ID NO 8.

PEG-bis-SS-pyridyl (2 mg dissolved in 1 mL water) was mixed with peptide SEQ ID NO 8 (3 mg dissolved in 0.1 mL methanol). The mixture was stirred and its UV absorbance at 340 nm was monitored. After 24 hrs the mixture was loaded onto a Vydac C18 preparative column (25×2.25 cm). The column was eluted with a two-component eluent gradient 0.5% per minute, starting from 0% of solution B in solution A, at flow rate 5 mL/min. Solution A was 0.1% TFA in H₂O, and solution B was 0.1% TFA in CH₃CN. Fractions 110-120 mL after the void volume were pooled together and freeze-dried.

### Example 17

Conjugation of Peptides SEQ ID NO 8 with PEI-PEG

PEI-PEG conjugate was prepared from polyethyleneimine MW ca. 2000 (PEI) and polyethylene glycol MW ca. 8000 (PEG) using carbonyldiimidazol (CDI) as previously described in Kabanov, A. V et al., Bioconjugate Chemistry 6, 639-643, 1995. PEI-PEG conjugate (10 mg dissolved in 0.2 mL phosphate buffer pH 8) was mixed with SPDP (3 mg from Sigma, dissolved in 0.05 mL dimethylformamide,) for 30 minutes. Then the mixture was applied onto a Sephadex G-25 column (Fisher, 20 ml) and eluted with 50 ml of phosphate buffer, and detect with UV absorption at 280 nm. The fraction containing PEI-PEG modified with SS-pyridyl was identified by increased UV absorption at 340 nm after mixing a sample with 1% ethanedithiol in methanol (1:1 v/v). This fraction (2 mL) was mixed with peptide SEQ ID NO 8 (2.5 mg dissolved in 0.05 mL DMF), and was stirred for 18 hrs. UV absorbance of the mixture at 340 nm was monitored. Acetic anhydride (0.005 mL) was added to the mixture, and it was stirred for 3 hrs. Then the mixture was dialyzed through cellulose membrane (Spectra/Por 1, MWCO 6000-8000) against water for 48 hrs, and freeze-dried.

### Example 18

Synthesis of Conjugate: paclitaxel-PEG-peptide SEQ ID NO 11

Peptide SEQ ID NO 11 was conjugated with Fmoc-NH-PEG-COO—N-succinimidyl (MW 3400 from Shearwater Polymers, Inc. Al., Cat. No. 1P2ZOF02), followed by Fmoc group removal with piperidin (step 1). The product was purified by HPLC. Paclitaxel-2′-succinate was obtained by the method described by Deutsch H. M. et al. (1989), J. Med. Chem. 32, 788-792. Paclitaxel-2′-succinate was conjugated with NH-PEG-peptide (SEQ ID NO 11) by means of EEDQ (2-ethoxy-1-ethoxycarbonyl-1,2-dihydroquinoline, the method was described by Safavy A. et al. (1999) J. Med. Chem. 42, 4919-4924), and produced paclitaxel-PEG-peptide(SEQ ID NO 11) (step 2).

1. Synthesis of Amino-PEG-Peptide (SEQ ID NO 11)

Peptide SEQ ID NO 11 (5 mg dissolved in 2 mL methanol) was mixed with Fmoc-NH-PEG-COO-N-succinimidyl (15 mg dissolved in 1 mL methanol) and diisopropylethylamine (0.01 mL of 10% solution in methanol). The mixture was stirred for 120 minutes, and then the solvent was removed in vacuum. The residue was dissolved in piperidine (1 mL of 20% solution in dimethylformamide), and it was mixed for 20 minutes. The mixture was then condensed in vacuum to 0.3 mL, and was diluted with 1 mL 50% aqueous acetonitrile, and it was loaded onto a Vydac C18 preparative column (25×2.25 cm). The column was eluted with a two-component eluent gradient 0.5% per minute, starting from 10% of solution B in solution A, at flow rate 5 mL/min. Solution A was 0.1% TFA in H₂O, and solution B was 0.1% TFA in CH₃CN. Fractions were identified by electrospray MS, pooled together and freeze dried.

2. Synthesis of Conjugate Paclitaxel-PEG-Peptide (SEQ ID NO 11)

Paclitaxel-2′-succinate (2 mg dissolved in 0.2 mL dimethylformamide) and EEDQ (1 mg) were mixed for 30 minutes. Then amino-PEG-peptide (SEQ ID NO 11) (3 mg in 0.2 mL dimethylformamide) was added, and the mixture was stirred for 3 hrs. The product was purified on Vydac C18 preparative column (25×2.25 cm) by elution with a two-component eluent gradient 0.5% per minute, starting from 10% of solution B in solution A, at flow rate 5 mL/min. Solution A was 0.1% TFA in H₂O, and solution B was 0.1% TFA in CH₃CN. Fractions were identified by electro-spray MS, pooled together and freeze dried.

### Example 19

Synthesis of Conjugate Paclitaxel-Polyglutamic Acid-Peptide SEQ ID NO 11

Paclitaxel-polyglutamic acid (paclitaxel-PG) was prepared from polyglutamic acid (PG) and paclitaxel using dicyclohexylcarbodiimide (DCC) in dimethylformamide (step 1). Paclitaxel-polyglutamic acid-peptide (SEQ ID NO 11) was prepared from paclitaxel-PG and peptide (SEQ ID NO 11) also using dicyclohexylcarbodiimide in dimethylformamide.

1. Synthesis of paclitaxel-polyglutamic acid (paclitaxel-PG)

Polyglutamic acid sodium salt (MW 50 K, Sigma, 0.5 g) was dissolved in water. The pH of the aqueous solution was adjusted to 2 using 0.2 M HCl. The precipitate was collected, dialyzed against distilled water, and lyophilized to yield 0.44 g PG. Paclitaxel (10 mg, 0.011 mmol), dicyclohexylcarbodiimide (5 mg, 0.025 mmol) and dimethylaminopyridine (0.1 mg) were added to a solution of polyglutamic acid (100 mg, 0.6 mmol of Glu residues) in anhydrous dimethylformamide (2 mL). The reaction was allowed to proceed at room temperature for 12 hrs. The reaction was controlled by thin layer chromatography on silica gel plates, using chlorophorm/MeOH (10:1) After 12 hours chromatography showed complete conversion of paclitaxel (Rf=0.5) to polymer conjugate (Rf=0). The reaction mixture was poured into chloroform. The precipitate was collected and dried in vacuum, dissolved in distilled water and freeze-dried.

2. Synthesis of paclitaxel-polyglutamic acid-peptide (SEQ ID NO 11)

Paclitaxel-polyglutamic acid (0.1 g) was dissolved in anhydrous dimethylformamide (2 mL), and dicyclohexylcarbodiimide (3 mg) was added, and stirred gently for 5 minutes. Then peptide SEQ ID NO 11 (3 mg) was added, and the mixture was stirred for 12 hours. Then the reaction mixture was poured into chloroform. The precipitate was collected and dried in vacuum, dissolved in distilled water and freeze-dried.

### Example 20

Synthesis of Conjugate Paclitaxel-Peptide SEQ ID NO 11

Paclitaxel-2′-succinate was obtained by the method described by Deutsch H. M. et al. (1989), J. Med. Chem. 32, 788-792. Paclitaxel-2′-succinate was conjugated with peptide (SEQ ID NO 11) by means of EEDQ (2-ethoxy-1-ethoxycarbonyl-1,2-dihydroquinoline, the method was described for example by Safavy A. et al. (1999) J. Med. Chem. 42, 4919-4924), and produced conjugate paclitaxel-peptide SEQ ID NO 11. The product was purified by HPLC.

Paclitaxel-2′-succinate (2 mg dissolved in 0.2 mL dimethylformamide) and EEDQ (1 mg) were mixed for 30 minutes. Then peptide (SEQ ID NO 11) (3 mg in 0.2 mL dimethylformamide) was added, and the mixture was stirred for 3 hrs. The product was purified on Vydac C18 preparative column (25×2.25 cm) by elution with a two-component eluent gradient 0.5% per minute, starting from 10% of solution B in solution A, at flow rate 5 mL/min. Solution A was 0.1% TFA in H₂O, and solution B was 0.1% TFA in CH₃CN. Fractions were identified by electro-spray MS, and freeze-dried.

### Example 21

Synthesis of Conjugate of Peptide SEQ ID NO 8 with Leucyl-doxorubicin

Leucyl-doxorubicin was modified using the heterobifunctional linker SPDP to produce pyridyl-SS-propionylleucyl-doxorubicin (step 1). The conjugate of peptide (SEQ ID NO 8) with Leucyl-doxorubicin was prepared by the reaction of pyridyl-SS-propionylleucyl-doxorubicin with peptide SEQ ID NO 8 (step 2).

1. Synthesis of pyridyl-SS-propionylleucyl-doxorubicin

Solution of L-leucyl-doxorubicin (6 mg, 0.01 mmol) in 0.1 mL methanol was mixed with SPDP (3 mg) solution in 0.1 mL methanol, and diisopropylethylamine (0.015 mL of 10% solution in methanol). The mixture was stirred for 20 minutes. The mixture was fractionated using reverse phase HPLC with water—acetonitrile gradient (1% per minute, starting from 10% acetonitrile). The fractions containing the product pyridyl-SS-propionylleucyl-doxorubicin were identified by MS (m/z 854).

2. Synthesis of Conjugate of Peptide SEQ ID NO 8 with propionylleucyl-doxorubicin

Pyridyl-SS-propionylleucyl-doxorubicin (1 mg dissolved in 0.1 mL dimethylformamide) was mixed with peptide SEQ ID NO 8 (2 mg dissolved in 0.02 mL DMF), and was stirred for 18 hrs. Then the mixture was diluted with 2 mL water and freeze-dried. The obtained material was purified with using reverse phase HPLC with water—acetonitrile gradient (1% per minute, starting from 10% acetonitrile). The fractions containing the conjugate were identified by MS.

FIG. 1. Formation S100A4 aggregates and their dissociation.

S100A4 aggregation was performed in TBS, containing either 5 mM Ca⁺⁺ or 5 mM EGTA for overnight at 37° C. To dissociate the S100A4 aggegates, they were treated at 37C° overnight with 5 mM DTT; 5 mM EGTA, or their combination. The resulted mixtures were analyzed by 15% SDS PAAG. 1) MW markers (New England BioLabs); 2) S100A4 monomer (control); 3) S100A4 aggregates in 5 mM Ca⁺⁺ (S100A4-D/Ca); 4) S100A4 aggregates in 5 mM EGTA (S100A4-D/E); 5) S100A4-D/Ca in presence of 5 mM DTT, 5 mM Ca⁺⁺; 6) S100A4-D/E in presence of 5 mM DTT; 7) S100A4-D/E in presence of 5 mM DTT, 5 mM EGTA; 8) S100A4-DIE, 5 mM EGTA; 9) S100A4-D/Ca in presence of 5 mM DTT, 5 mM EGTA; 10) S100A4-D/Ca, 5 mM EGTA.

FIG. 2. Immuno-co precipitation of Annexin II by Mtsl.

Annexin II/S100A4 complex (S100A4 monomer, S100A4-D/Ca or S100A4-D/E) was precipitated by rabbit anti-S100A4 antibody using protein G-Sepharose and subjected to 10% SDS gel electrophoresis in reducing conditions followed by Western blot using anti-Annexin II antibody. 1) Annexin II; 2) IP S100A4/Annexin II ab/Ca; 3) IP S100A4/Annexin II/Annexin II ab/Ca; 4) IP S100A4/Annexin II/Annexin II ab/EGTA; 5) IP S100A4/Annexin II ab/EGTA; 6) IP S100A4-D/Ca/Annexin II/Annexin II ab/Ca; 7) IP S100A4-D/Ca/Annexin II ab/Ca; 8) IP S100A4-D/Ca/Annexin II/Annexin II ab/EGTA. 9) Annexin II, S100A4-D/E, 5 mM Ca⁺⁺; 10) Annexin II, S100A4-D/E, 5 mM Ca⁺⁺, 5 mM DTT; 11) Annexin II, S100A4-D/E, 5 mM EGTA 12) Annexin II, S100A4-D/E, 5 mM DTT.

FIG. 3. Surface plasmon resonance sensograms of S100A4 interaction with immobilized Annexin 2 in HBS buffer with 3.5 mM EDTA.

FIG. 4. Surface plasmon resonance sensograms of S100A4 interaction with immobilized Annexin 2 in HBS buffer with 1 mM CaCl₂.

FIG. 5. Change of fluorescence of 5 micromol/L solutions of peptides SEQ ID NO 7, 9, 10, 13, and 15 upon interaction with S100A4 at concentration 3 micromol/L.

FIG. 6. Change of fluorescence polarization of 20 nanomol/L solutions of peptides SEQ ID NO 7 upon interaction with S100A4.

FIG. 7. Inhibition of S100A4 induced capillary-like tube formation with peptide SEQ ID NO 6 in HCEC culture.

1. An isolated polypeptide selected from the group consisting of:
(a) an amino acid sequence X¹X²ERX³, wherein X¹ is selected from the group consisting of Y and F; X² is selected from the group consisting of M, V, L, I; and X³ is selected from the group consisting of W, V, L, I, F, H; or a derivative thereof; and
(b) an amino acid sequence X¹RE X²X³, wherein X¹ is selected from the group consisting of W, V, L, I, F, H; X² is selected from the group consisting of M, V, L, I; and X³ is selected from the group consisting of Y and F; or a derivative thereof.

2. The isolated polypeptide of claim 1 where at least one of the amino acids is substituted by its D-enantiomer.

3. The isolated polypeptide of claim 1 wherein the amino acid sequence is selected from the group consisting of SEQ ID NO:12, SEQ ID NO:14 and SEQ ID NOS: 41 to 87.

4. The isolated polypeptide of claim 2 selected from the group consisting of SEQ ID NOS: 33 to 36.

5. The isolated polypeptide of claim 1 which specifically binds to S100A4.

6. A compound comprising at least two polypeptides according to claim 1 conjoined with a chemical linker, the chemical linker being between approximately 5 Angstroms and approximately 100 Angstroms.

7. The compound of claim 6 where the chemical linker comprises a linear chain of 18 atoms.

8. The compound of claim 7, where the chemical linker comprises amino acids.

9. The compound of claim 8 comprising a polypeptide selected from the group consisting of SEQ ID NO:5 and SEQ ID NO:37.

10. The compound of claim 6 wherein a first molecule is conjoined to the compound.

11. A compound of claim 10 wherein the first molecule is selected from the group consisting of a protein, a polymer and a therapeutic agent.

12. A compound of claim 11, wherein a second molecule is conjoined to the compound.

13. A compound of claim 12 wherein the second molecule is a therapeutic agent.

14. A composition comprising a polypeptide of claim 1 and a pharmaceutically or physiologically acceptable carrier.

15. The composition of claim 14 wherein the carrier is comprised of one or more nonionic water-soluble, nonionic hydrophobic or poorly water soluble, cationic, anionic or polyampholite polymeric carriers.

16. The composition of claim 15, wherein said composition further comprises a biological agent.

17. The composition of claim 16 wherein the biological agent comprises a protein, peptide, recombinant soluble receptor, monoclonal antibody, human growth hormone, tissue plasminogen activator, clotting factor, vaccine, colony stimulating factor, erythropoietin, enzyme, dismultase anti-neoplastic agent, antibacterial agent, antiparasitic agent, anti-fungal agent, CNS agent, immunomodulator and cytokine, toxin, neuropeptide or modified biological agent.

18. The composition of claim 17, wherein said modified biological agent further comprises a pro-drug.

19. The composition of claim 15, wherein said composition further comprises a therapeutic agent.

20. A method of treating a disease associated with activated endothelium in a patient in need of such therapy comprising administering to said patient a therapeutically effective amount of said composition of claim 14.

21. An isolated nucleic acid encoding the amino acid sequences of claim 1.

22. An isolated nucleic acid encoding the amino acid sequences of SEQ ID NO: 5.

23. The isolated nucleic acid of claim 21 encoding an amino acid sequence selected from the group consisting of SEQ ID NO:12, SEQ ID NO:14 and SEQ ID NOS: 41 to 87.

24. An expression vector comprising the nucleic acid sequence of claim 21.

25. A host cell transfected with the expression vector of claim 24.

26. A composition comprising a nucleic acid molecule of claim 21 and a pharmaceutically or physiologically acceptable carrier.

27. The composition of claim 16 wherein said biological agent is beta-galactosidase or a therapeutic protein, and said biological agent is genetically or chemically fused with a polypeptide capable of specific binding with S100A4.

28. The composition of claim 16 wherein at least one of the polypeptides is capable of specific binding with S100A4 and the at least one of the polypeptides is conjugated with one or more nonionic water-soluble, nonionic hydrophobic or poorly water soluble, cationic, anionic or polyampholite polymers.

29. A composition according to claim 28 wherein the at least one of the polypeptides or the polymer is conjugated with a biological agent.

30. A pharmaceutical composition according to claim 29 wherein said biological agent is conjugated with the at least one of the polypeptides.
